(19) United States
(12) Patent Application Publication
(10) Pub. No.: US 2004/0006073 A1
(43) Pub. Date: Jan. 8, 2004

(54) Method of treating attention deficit hyperactivity disorder

(76) Inventor: David James Dooley, South Lyon, MI (US)

Correspondence Address: David R. Kurlandsky, Warner-Lambert Company LLC, 2800 Plymouth Road, Ann Arbor, MI, 48105, US

(21) Appl. No.: 10/602,414

(22) Filed: Jun. 23, 2003

Related U.S. Application Data
(60) Provisional application No. 60/392,140, filed on Jun. 27, 2002.

(51) Int. Cl. A61K 31/551

(52) U.S. Cl. 514/220

(57) ABSTRACT

This invention relates to a method of treating ADHD by administering an alpha2delta ligand such as, for example, gabapentin or pregabalin, or a pharmaceutically acceptable salt thereof.

This application claims the benefit of priority to U.S. Provisional Application No. 60/392,140 filed Jun. 27, 2002.

This invention relates to a method of preventing or treating attention deficit hyperactivity disorder (“ADHD”) by administering a compound that exhibits activity as an alpha2delta ligand (α2δ ligand). Such compounds have affinity for the α2δ subunit of a calcium channel. Such compounds have also been referred to in the literature as gamma-aminobutyric acid (GABA) analogs.

### BACKGROUND OF THE INVENTION

Attention deficit hyperactivity disorder (ADHD) has an estimated incidence in school age children of 3-5%. The attentional symptoms of ADHD can be successfully treated with psychomotor stimulants such as methylphenidate (Ritalin). Clonidine, an α₂-adrenoceptor agonist, treats the aggressive and oppositional symptoms. There is a potential for significant side effects with both methylphenidate and clonidine, making it important to identify other drugs that have similar or better efficacy with reduced side effects.

Since ADHD can be characterized as a dysregulation of catecholaminergic neurotransmission in executive brain regions like prefrontal cortex, it is possible that drugs acting to modulate this neurotransmission may be of potential relevance to treat ADHD. In this regard, alpha₂delta ligands including gabapentin and pregabalin may be efficacious in treating this disorder. This hypothesis is based on our previous observation that gabapentin and pregabalin appear to preferentially attenuate neurotransmitter release induced by stimuli considered pathological in nature (J. Pharmacol. Exp. Ther. 295:1086-1093, 2000). Therefore, ADHD may also be an indication sensitive to alpha₂delta ligands either alone or in combination with stimulants (e.g., Ritalin) or non-stimulants (e.g., atomoxetine, GT-2331 (Perceptin)).

ADHD is one of the most common childhood psychiatric disorders and appears to be a common, often underrecognized, psychiatric disease in adults as well (Spencer T, 1998). This disorder, which begins in childhood, may be followed by a lifelong expression of symptoms (e.g., hyperactivity and/or impulsivity) (Schweitzer J B, 2001). ADHD may change its manifestations as it develops from preschool through adult life (Cantwell D P, 1996; Elia J, 1999; Nolan E E, 2001).

The diagnosis of ADHD is based on clinical evaluation (Dulcan M, 1997; National Institutes of Health, 1998). “The essential feature of ADHD is a persistent pattern of inattention and/or hyperactivity-impulsivity that is more frequent and severe than is typically observed in individuals at a comparative level of development” (Diagnostic and Statistical Manual of Mental Disorders (DSM-IV), American Psychiatric Association, Washington, D.C., 1994). In order to be diagnosed with ADHD, patients must demonstrate symptoms of ADHD that cause impairment before the age of seven years, and symptoms must have been ongoing for longer than six months in at least two settings (e.g., school [or work] and home). (See DSM-IV).

Several alpha2delta ligands are known. Gabapentin, a cyclic alpha2delta ligand, is now commercially available (Neurontin®, Warner-Lambert Company) and extensively used clinically for treatment of epilepsy and neuropathic pain. Such cyclic alpha2delta ligands are described in U.S. Pat. No. 4,024,175, which issued on May 17, 1977, and U.S. Pat. No. 4,087,544, which issued on May 2, 1978. Other series of alpha2delta ligands are described in U.S. Pat. No. 5,563,175, which issued on Oct. 8, 1996, U.S. Pat. No. 6,316,638, which issued on Nov. 13, 2001, U.S. Provisional Patent Application 60/353,632, which was filed on Jan. 31, 2002, European Patent Application EP 1112253, which was published on Jul. 4, 2001, PCT Patent Application WO 99/08671, which was published on Feb. 25, 1999, and PCT Patent Application WO 99/61424, which was published on Dec. 2, 1999. These patents and applications are incorporated herein by reference in their entireties.

### SUMMARY OF THE INVENTION

This invention provides a method of preventing or treating ADHD in a mammal suffering therefrom, comprising administering a therapeutically effective amount of an alpha2delta ligand or a pharmaceutically acceptable salt thereof. The foregoing method is sometimes referred to herein as the “invention method”.

A preferred embodiment of the invention method utilizes an alpha2delta ligand that is a cyclic amino acid compound of Formula I

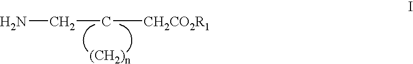

wherein R₁ is hydrogen or lower alkyl and n is an integer of from 4 to 6, and the pharmaceutically acceptable salts thereof. An especially preferred embodiment utilizes a compound of Formula I where R₁ is hydrogen and n is 5, which compound is 1-(aminomethyl)-cyclohexane acetic acid, known generically as gabapentin. Other preferred alpha2delta ligands, or a pharmaceutically acceptable salt thereof, are compounds of Formula I wherein the cyclic ring is substituted, for example with alkyl such as methyl or ethyl. Typical of such compounds include (1-aminomethyl-3-methylcyclohexyl) acetic acid, (1-aminomethyl-3-methylcyclopentyl) acetic acid, and (1-aminomethyl-3,4-dimethylcyclopentyl) acetic acid.

In another preferred embodiment, the invention method utilizes an alpha2delta ligand of Formula II

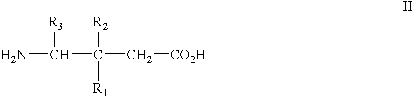

or a pharmaceutically acceptable salt thereof, wherein:

R₁ is a straight or branched unsubstituted alkyl of from 1 to 6 carbon atoms, unsubstituted phenyl, or unsubstituted cycloalkyl of from 3 to 6 carbon atoms;

R₂ is hydrogen or methyl; and

R₃ is hydrogen, methyl, or carboxyl.

Diastereomers and enantiomers of compounds of Formula II can be utilized in the invention method.

An especially preferred embodiment of the invention method employs a compound of Formula II where R₂ and R₃ are both hydrogen, and R₁ is —(CH₂)₀₋₂-i C₄H₉ as an (R), (S), or (R,S) isomer.

A more preferred embodiment of the invention method utilizes a compound of Formula II named 3-aminomethyl-5-methyl-hexanoic acid, or especially (S)-3-(aminomethyl)-5-methylhexanoic acid, now known generically as pregabalin. Pregabalin is also known as “CI-1008” and “S-(+)-3-IBG.”

Another preferred embodiment of the invention method utilizes a compound of Formula II named 3-(1-aminoethyl)-5-methylheptanoic acid or 3-(1-aminoethyl)-5-methylhexanoic acid.

Another preferred embodiment of the invention method utilizes an alpha2delta ligand that is a compound of the Formula III, IIIC, IIIF, IIIG, or IIIH

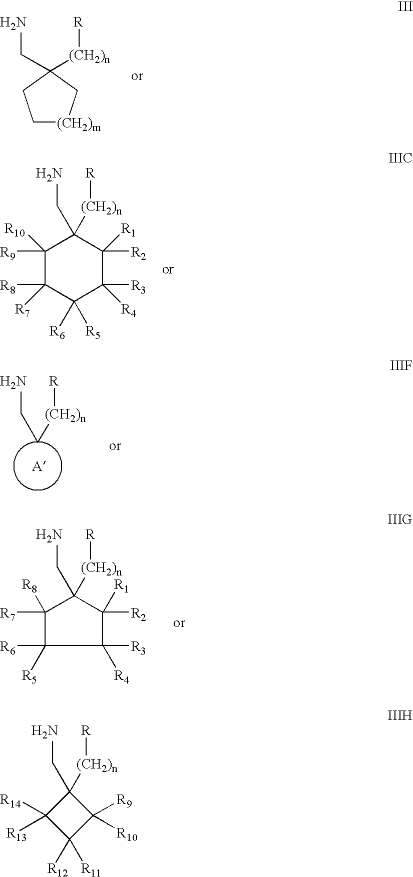

or a pharmaceutically acceptable salt thereof wherein:

n is an integer of from 0 to 2;

m is an integer of from 0 to 3;

R is sulfonamide,

amide,

phosphonic acid,

heterocycle,

sulfonic acid, or

hydroxamic acid;

with the proviso that R can not be sulfonic acid when m is 2 and n is 1;

R₁ to R₁₄ are each independently selected from hydrogen or straight or branched alkyl of from 1 to 6 carbons, unsubstituted or substituted benzyl or phenyl which substituents are selected from halogen, alkyl, alkoxy, hydroxy, carboxy, carboalkoxy, trifluoromethyl, and nitro;

A′ is a bridged ring selected from

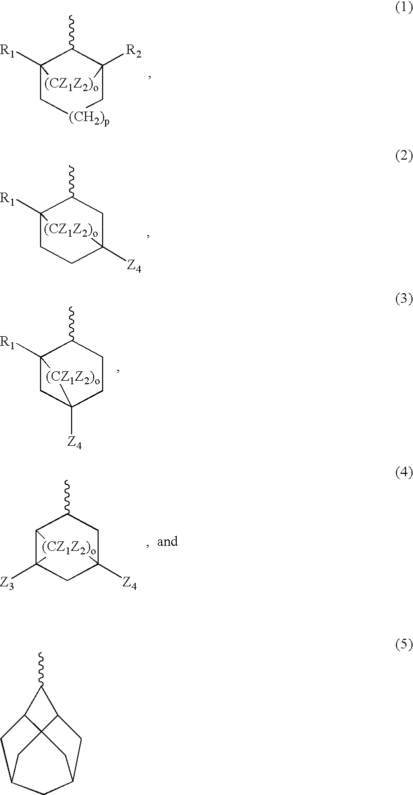

wherein

[symbol] is the point of attachment;

Z₁ to Z₄ are each independently selected from hydrogen and methyl;

o is an integer of from 1 to 4; and

p is an integer of from 0 to 2.

Another preferred embodiment of the invention method utilizes a compound of Formulas III, IIIC, IIIF, IIIG, or IIIH selected from:

(1-Aminomethyl-cyclohexylmethyl)-phosphonic acid;

(1R-trans)(1-Aminomethyl-3-methyl-cyclohexylmethyl)-phosphonic acid;

(trans)(1-Aminomethyl-3,4-dimethyl-cyclopentylmethyl)-phosphonic acid;

(1R-trans)(1-Aminomethyl-3-methyl-cyclopentylmethyl)-phosphonic acid;

(1S-cis)(1-Aminomethyl-3-methyl-cyclopentylmethyl)-phosphonic acid;

(1S-trans)(1-Aminomethyl-3-methyl-cyclopentylmethyl)-phosphonic acid;

(1R-cis)(1-Aminomethyl-3-methyl-cyclopentylmethyl)-phosphonic acid;

(1α,3α,4α)(1-Aminomethyl-3,4-dimethyl-cyclopentylmethyl)-phosphonic acid;

(1α,3β,4β)(1-Aminomethyl-3,4-dimethyl-cyclopentylmethyl)-phosphonic acid;

(R)(1-Aminomethyl-3,3-dimethyl-cyclopentylmethyl)-phosphonic acid;

(S)(1-Aminomethyl-3,3-dimethyl-cyclopentylmethyl)-phosphonic acid;

(1-Aminomethyl-3,3-dimethyl-cyclobutylmethyl)-phosphonic acid;

2-(1-Aminomethyl-cyclohexyl)-N-hydroxy-acetamide;

(1S-trans)2-(1-Aminomethyl-3-methyl-cyclohexyl)-N-hydroxy-acetamide;

(trans)2-(1-Aminomethyl-3,4-dimethyl-cyclopentyl)-N-hydroxy-acetamide;

(1S-cis)2-(1-Aminomethyl-3-methyl-cyclopentyl)-N-hydroxy-acetamide;

(1R-trans)2-(1-Aminomethyl-3-methyl-cyclopentyl)-N-hydroxy-acetamide;

(1R-cis)2-(1-Aminomethyl-3-methyl-cyclopentyl)-N-hydroxy-acetamide;

(1S-trans)2-(1-Aminomethyl-3-methyl-cyclopentyl)-N-hydroxy-acetamide;

(1α,3α,4α)2-(1-Aminomethyl-3,4-dimethyl-cyclopentyl)-N-hydroxy-acetamide;

(1α,3β,4β)2-(1-Aminomethyl-3,4-dimethyl-cyclopentyl)-N-hydroxy-acetamide;

(S)2-(1-Aminomethyl-3,3-dimethyl-cyclopentyl)-N-hydroxy-acetamide;

(R)2-(1-Aminomethyl-3,3-dimethyl-cyclopentyl)-N-hydroxy-acetamide;

2-(1-Aminomethyl-3,3-dimethyl-cyclobutyl)-N-hydroxy-acetamide;

N-[2-(1-Aminomethyl-cyclohexyl)-ethyl]-methanesulfonamide;

(1S-cis)N-[2-(1-Aminomethyl-3-methyl-cyclohexyl)-ethyl]-methanesulfonamide;

(trans)N-[2-(1-Aminomethyl-3,4-dimethyl-cyclopentyl)-ethyl]-methanesulfonamide;

(1S-cis)N-[2-(1-Aminomethyl-3-methyl-cyclopentyl)-ethyl]-methanesulfonamide;

(1R-trans)N-[2-(1-Aminomethyl-3-methyl-cyclopentyl)-ethyl]-methanesulfonamide;

(1R-cis)N-[2-(1-Aminomethyl-3-methyl-cyclopentyl)-ethyl]-methanesulfonamide;

(1S-cis)N-[2-(1-Aminomethyl-3-methyl-cyclopentyl)-ethyl]-methanesulfonamide;

(1α,3α,4α)N-[2-(1-Aminomethyl-3,4-dimethyl-cyclopentyl)-ethyl]-methanesulfonamide;

(1α,3β,4β)N-[2-(1-Aminomethyl-3,4-dimethyl-cyclopentyl)-ethyl]-methanesulfonamide;

(S)N-[2-(1-Aminomethyl-3,3-dimethyl-cyclopentyl)-ethyl]-methanesulfonamide;

(R)N-[2-(1-Aminomethyl-3,3-dimethyl-cyclopentyl)-ethyl]-methanesulfonamide;

N-[2-(1-Aminomethyl-3,3-dimethyl-cyclobutyl)-ethyl]-methanesulfonamide;

(1S-cis)3-(1-Aminomethyl-3-methyl-cyclohexylmethyl)-4H-[1,2,4]oxadiazol-5-one;

(trans)3-(1-Aminomethyl-3,4-dimethyl-cyclopentylmethyl)-4H-[1,2,4]oxadiazol-5-one;

(1S-cis)3-(1-Aminomethyl-3-methyl-cyclopentylmethyl)-4H-[1,2,4]oxadiazol-5-one;

(1R-trans)3-(1-Aminomethyl-3-methyl-cyclopentyl methyl)-4H-[1,2,4]oxadiazol-5-one;

(1R-cis)3-(1-Aminomethyl-3-methyl-cyclopentylmethyl)-4H-[1,2,4]oxadiazol-5-one;

(1S-trans)3-(1-Aminomethyl-3-methyl-cyclopentylmethyl)-4H-[1,2,4]oxadiazol-5-one;

(1α,3α,4α)3-(1-Aminomethyl-3,4-dimethyl-cyclopentylmethyl)-4H-[1,2,4]oxadiazol-5-one;

(1α,3β,4β)3-(1-Aminomethyl-3,4-dimethyl-cyclopentylmethyl)-4H-[1,2,4]oxadiazol-5-one;

(S)3-(1-Aminomethyl-3,3-dimethyl-cyclopentylmethyl)-4H-[1,2,4]oxadiazol-5-one;

(R)3-(1-Aminomethyl-3,3-dimethyl-cyclopentylmethyl)-4H-[1,2,4]oxadiazol-5-one;

3-(1-Aminomethyl-3,3-dimethyl-cyclobutylmethyl)-4H-[1,2,4]oxadiazol-5-one;

3-(1-Aminomethyl-cyclohexylmethyl)-4H-[1,2,4]oxadiazole-5-thione;

(1S-cis)3-(1-Aminomethyl-3-methyl-cyclohexylmethyl)-4H-[1,2,4]oxadiazole-5-thione;

(trans)3-(1-Aminomethyl-3,4-dimethyl-cyclopentylmethyl)-4H-[1,2,4]oxadiazole-5-thione;

(1S-cis)3-(1-Aminomethyl-3-methyl-cyclopentylmethyl)-4H-[1,2,4]oxadiazole-5-thione;

(1R-trans)3-(1-Aminomethyl-3-methyl-cyclopentylmethyl)-4H-[1,2,4]oxadiazole-5-thione;

(1R-cis)3-(1-Aminomethyl-3-methyl-cyclopentylmethyl)-4H-[1,2,4]oxadiazole-5-thione;

(1S-trans)3-(1-Aminomethyl-3-methyl-cyclopentylmethyl)-4H-[1,2,4]oxadiazole-5-thione;

(1α,3α,4α)3-(1-Aminomethyl-3,4-dimethyl-cyclopentylmethyl)-4H-[1,2,4]oxadiazole-5-thione;

(1α,3α,4α)3-(1-Aminomethyl-3,4-dimethyl-cyclopentylmethyl)-4H-[1,2,4]oxadiazole-5-thione;

(S)3-(1-Aminomethyl-3,3-dimethyl-cyclopentyl methyl)-4H-[1,2,4]oxadiazole-5-thione;

(R)3-(1-Aminomethyl-3,3-dimethyl-cyclopentylmethyl)-4H-[1,2,4]oxadiazole-5-thione;

3-(1-Aminomethyl-3,3-dimethyl-cyclobutylmethyl)-4H-[1,2,4]oxadiazole-5-thione;

C-[1-(1H-Tetrazol-5-ylmethyl)-cyclohexyl]-methylamine;

(1S-cis)C-[3-Methyl-1-(1H-tetrazol-5-ylmethyl)-cyclohexyl]-methylamine;

(trans)C-[3,4-Dimethyl-1-(1H-tetrazol-5-ylmethyl)-cyclopentyl]-methylamine;

(1S-cis)C-[3-Methyl-1-(1H-tetrazol-5-ylmethyl)-cyclopentyl]-methylamine;

(1R-trans)C-[3-Methyl-1-(1H-tetrazol-5-ylmethyl)-cyclopentyl]-methylamine;

(1R-cis)C-[3-Methyl-1-(1H-tetrazol-5-ylmethyl)-cyclopentyl]-methylamine;

(1S-trans)C-[3-Methyl-1-(1H-tetrazol-5-ylmethyl)-cyclopentyl]-methylamine;

(1α,3α,4α)C-[3,4-Dimethyl-1(1H-tetrazol-5-ylmethyl)-cyclopentyl]-methylamine;

(1α,3β,4β)C-[3,4-Dimethyl-1-(1H-tetrazol-5-ylmethyl)-cyclopentyl]-methylamine;

(S)C-[3,3-Dimethyl-1-(1H-tetrazol-5-ylmethyl)-cyclopentyl]-methylamine;

(R)C-[3,3-Dimethyl-1(1H-tetrazol-5-ylmethyl)-cyclopentyl]-methylamine;

C-[3,3-Dimethyl-1-(1H-tetrazol-5-ylmethyl)-cyclobutyl]-methylamine;

N-[2-(1-Aminomethyl-cyclohexyl)-ethyl]-C,C,C-trifluoro-methanesulfonamide;

(1S-cis)N-[2-(1-Aminomethyl-3-methyl-cyclohexyl)-ethyl]-C,C,C-trifluoro-methanesulfonamide;

(trans)N-[2-(1-Aminomethyl-3,4-dimethyl-cyclopentyl)-ethyl]-C,C,C-trifluoro-methanesulfonamide;

(1R-cis)N-[2-(1-Aminomethyl-3-methyl-cyclopentyl)-ethyl]-C,C,C-trifluoro-methanesulfonamide;

(1S-trans)N-[2-(1-Aminomethyl-3-methyl-cyclopentyl)-ethyl]-C,C,C-trifluoro-methanesulfonamide;

(1S-cis)N-[2-(1-Aminomethyl-3-methyl-cyclopentyl)-ethyl]-C,C,C-trifluoro-methanesulfonamide;

(1R-trans)N-[2-(1-Aminomethyl-3-methyl-cyclopentyl)-ethyl]-C,C,C-trifluoro-methanesulfonamide;

(1α,3α,4α)N-[2-(1-Aminomethyl-3,4-dimethyl-cyclopentyl)-ethyl]-C,C,C-trifluoro-methanesulfonamide;

(1α,3β,4β)N-[2-(1-Aminomethyl-3,4-dimethyl-cyclopentyl)-ethyl]-C,C,C-trifluoro-methanesulfonamide;

(S)N-[2-(1-Aminomethyl-3,3-dimethyl-cyclopentyl)-ethyl]-C,C,C-trifluoro-methanesulfonamide;

(R)N-[2-(1-Aminomethyl-3,3-dimethyl-cyclopentyl)-ethyl]-C,C,C-trifluoro-methanesulfonamide;

N-[2-(1-Aminomethyl-3,3-dimethyl-cyclobutyl)-ethyl]-C,C,C-trifluoro-methanesulfonamide;

3-(1-Aminomethyl-cyclohexylmethyl)-4H-[1,2,4]thiadiazol-5-one;

(1S-cis)3-(1-Aminomethyl-3-methyl-cyclohexylmethyl)-4H-[1,2,4]thiadiazol-5-one;

(trans)3-(1-Aminomethyl-3,4-dimethyl-cyclopentylmethyl)-4H-[1,2,4]thiadiazol-5-one;

(1R-cis)3-(1-Aminomethyl-3-methyl-cyclopentylmethyl)-4H-[1,2,4]thiadiazol-5-one;

(1S-trans)3-(1-Aminomethyl-3-methyl-cyclopentylmethyl)-4H-[1,2,4]thiadiazol-5-one;

(1S-cis)3-(1-Aminomethyl-3-methyl-cyclopentylmethyl)-4H-[1,2,4]thiadiazol-5-one;

(1R-trans)3-(1-Aminomethyl-3-methyl-cyclopentylmethyl)-4H-[1,2,4]thiadiazol-5-one;

(1α,3α,4α)-3-(1-Aminomethyl-3,4-dimethyl-cyclopentylmethyl)-4H-[1,2,4]thiadiazol-5-one;

(1α,3α,4α)3-(1-Aminomethyl-3,4-dimethyl-cyclopentylmethyl)-4H-[1,2,4]thiadiazol-5-one;

(S)3-(1-Aminomethyl-3,3-dimethyl-cyclopentylmethyl)-4H-[1,2,4]thiadiazol-5-one;

(R)3-(1-Aminomethyl-3,3-dimethyl-cyclopentylmethyl)-4H-[1,2,4]thiadiazol-5-one;

3-(1-Aminomethyl-3,3-dimethyl-cyclobutylmethyl)-4H-[1,2,4]thiadiazol-5-one;

C-[1-(2-Oxo-2,3-dihydro-2λ⁴-[1,2,3,5]oxathiadiazol-4-ylmethyl)-cyclohexyl]-methylamine;

(1S-cis)C-[3-Methyl-1-(2-oxo-2,3-dihydro-2,4-[1,2,3,5]oxathiadiazol-4-ylmethyl)-cyclohexyl]-methyl amine;

(trans)C-[3,4-Dimethyl-1-(2-oxo-2,3-dihydro-2λ⁴-[1,2,3,5]oxathiadiazol-4-ylmethyl)-cyclopentyl]-methyl amine;

(1S-cis)C-[3-Methyl-1-(2-oxo-2,3-dihydro-2λ⁴-[1,2,3,5]oxathiadiazol-4-ylmethyl)-cyclopentyl]-methylamine;

(1R-trans)C-[3-Methyl-1-(2-oxo-2,3-dihydro-2,4-[1,2,3,5]oxathiadiazol-4-ylmethyl)-cyclopentyl]-methylamine;

(1R-cis)C-[3-Methyl-1-(2-oxo-2,3-dihydro-2λ⁴-[1,2,3,5]oxathiadiazol-4-ylmethyl)-cyclopentyl]-methylamine;

(1S-trans)C-[3-Methyl-1-(2-oxo-2,3-dihydro-2)⁴-[1,2,3,5]oxathiadiazol-4-ylmethyl)-cyclopentyl]-methylamine;

(1α,3α,4α)C-[3,4-Dimethyl-1-(2-oxo-2,3-dihydro-2λ⁴-[1,2,3,5]oxathiadiazol-4-ylmethyl)-cyclopentyl]-methylamine;

(1α,3α,4α)C-[3,4-Dimethyl-1-(2-oxo-2,3-dihydro-2λ⁴-[1,2,3,5]oxathiadiazol-4-ylmethyl)-cyclopentyl]-methylamine;

(S)C-[3,3-Dimethyl-1-(2-oxo-2,3-dihydro-2λ⁴-[1,2,3,5]oxathiadiazol-4-ylmethyl)-cyclopentyl]-methyl amine;

(R)C-[3,3-Dimethyl-1-(2-oxo-2,3-dihydro-2λ⁴-[1,2,3,5]oxathiadiazol-4-ylmethyl)-cyclopentyl]-methyl amine;

C-[3,3-Dimethyl-1-(2-oxo-2,3-dihydro-2λ⁴-[1,2,3,5]oxathiadiazol-4-ylmethyl)-cyclobutyl]-methylamine;

(1-Aminomethyl-cyclohexyl)-methanesulfonamide;

(1R-trans)(1-Aminomethyl-3-methyl-cyclohexyl)-methanesulfonamide;

(trans)(1-Aminomethyl-3,4-dimethyl-cyclopentyl)-methanesulfonamide;

(1S-trans)(1-Aminomethyl-3-methyl-cyclopentyl)-methanesulfonamide;

(1R-cis)(1-Aminomethyl-3-methyl-cyclopentyl)-methanesulfonamide;

(1R-trans)(1-Aminomethyl-3-methyl-cyclopentyl)-methanesulfonamide;

(1S-cis)(1-Aminomethyl-3-methyl-cyclopentyl)-methanesulfonamide;

(1α,3β,4β)(1-Aminomethyl-3,4-dimethyl-cyclopentyl)-methanesulfonamide;

(1α,3α,4α)(1-Aminomethyl-3,4-dimethyl-cyclopentyl)-methanesulfonamide;

(R)(1-Aminomethyl-3,3-dimethyl-cyclopentyl)-methanesulfonamide;

(S)(1-Aminomethyl-3,3-dimethyl-cyclopentyl)-methanesulfonamide;

(1-Aminomethyl-3,3-dimethyl-cyclobutyl)-methanesulfonamide;

(1-Aminomethyl-cyclohexyl)-methanesulfonic acid;

(1R-trans)(1-Aminomethyl-3-methyl-cyclohexyl)-methanesulfonic acid;

(trans)(1-Aminomethyl-3,4-dimethyl-cyclopentyl)-methanesulfonic acid;

(1S-trans)(1-Aminomethyl-3-methyl-cyclopentyl)-methanesulfonic acid;

(1S-cis)(1-Aminomethyl-3-methyl-cyclopentyl)-methanesulfonic acid;

(1R-trans)(1-Aminomethyl-3-methyl-cyclopentyl)-methanesulfonic acid;

(1R-cis)(1-Aminomethyl-3-methyl-cyclopentyl)-methanesulfonic acid;

(1α,3β,4β)(1-Aminomethyl-3,4-dimethyl-cyclopentyl)-methanesulfonic acid;

(1α,3α,4α)(1-Aminomethyl-3,4-dimethyl-cyclopentyl)-methanesulfonic acid;

(R)(1-Aminomethyl-3,3-dimethyl-cyclopentyl)-methanesulfonic acid;

(S)(1-Aminomethyl-3,3-dimethyl-cyclopentyl)-methanesulfonic acid;

(1-Aminomethyl-3,3-dimethyl-cyclobutyl)-methanesulfonic acid;

(1-Aminomethyl-cyclopentylmethyl)-phosphonic acid;

2-(1-Aminomethyl-cyclopentyl)-N-hydroxy-acetamide;

N-[2-(1-Aminomethyl-cyclopentyl)-ethyl]-methanesulfonamide;

3-(1-Aminomethyl-cyclopentylmethyl)-4H-[1,2,4]oxadiazol-5-one;

3-(1-Aminomethyl-cyclopentylmethyl)-4H-[1,2,4]oxadiazole-5-thione;

C-[1-(1H-Tetrazol-5-ylmethyl)-cyclopentyl]-methylamine;

N-[2-(1-Aminomethyl-cyclopentyl)-ethyl]-C,C,C-trifluoro-methanesulfonamide;

3-(1-Aminomethyl-cyclopentylmethyl)-4H-[1,2,4]thiadiazol-5-one;

C-[-(2-Oxo-2,3-dihydro-2λ⁴-[1,2,3,5]oxathiadiazol-4-ylmethyl)-cyclopentyl]-methylamine;

(1-Aminomethyl-cyclopentyl)-methanesulfonamide;

(1-Aminomethyl-cyclopentyl)-methanesulfonic acid;

(9-Aminomethyl-bicyclo[3.3.1]non-9-ylmethyl)-phosphonic acid;

2-(9-Aminomethyl-bicyclo[3.3.1]non-9-yl)-N-hydroxy-acetamide;

N-[2-(9-Aminomethyl-bicyclo[3.3.1]non-9-yl)-ethyl]-methanesulfonamide;

3-(9-Aminomethyl-bicyclo[3.3.1]non-9-ylmethyl)-4H-[1,2,4]oxadiazol-5-one;

3-(9-Aminomethyl-bicyclo[3.3.1]non-9-ylmethyl)-4H-[1,2,4]oxadiazole-5-thione;

C-[9-(1H-Tetrazol-5-ylmethyl)-bicyclo[3.3.1]non-9-yl]-methylamine;

N-[2-(9-Aminomethyl-bicyclo[3.3.1]non-9-yl)-ethyl]-C,C,C-trifluoro-methanesulfonamide;

3-(9-Aminomethyl-bicyclo[3.3.1]non-9-ylmethyl)-4H-[1,2,4] thiadiazol-5-one;

C-[9-(2-Oxo-2,3-dihydro-2λ⁴-[1,2,3,5]oxathiadiazol-4-ylmethyl)-bicyclo[3.3.1]non-9-yl]-methylamine;

(9-Aminomethyl-bicyclo[3.3.1]non-9-yl)-methanesulfonamide;

(9-Aminomethyl-bicyclo[3.3.1]non-9-yl)-methanesulfonic acid;

(2-Aminomethyl-adamantan-2-ylmethyl)-phosphonic acid;

2-(2-Aminomethyl-adamantan-2-yl)-N-hydroxy-acetamide;

N-[2-(2-Aminomethyl-adamantan-2-yl)-ethyl]-methanesulfonamide;

3-(2-Aminomethyl-adamantan-2-ylmethyl)-4H-[1,2,4]oxadiazol-5-one;

3-(2-Aminomethyl-adamantan-2-ylmethyl)-4H-[1,2,4]oxadiazole-5-thione;

C-[2-(1H-Tetrazol-5-ylmethyl)-adamantan-2-yl]-methylamine;

N-[2-(2-Aminomethyl-adamantan-2-yl)-ethyl]-C,C,C-trifluoro-methanesulfonamide;

3-(2-Aminomethyl-adamantan-2-ylmethyl)-4H-[1,2,4]thiadiazol-5-one;

C-[2-(2-Oxo-2,3-dihydro-2λ⁴-[1,2,3,5]oxathiadiazol-4-ylmethyl)-adamantan-2-yl]-methylamine;

(2-Aminomethyl-adamantan-2-yl)-methanesulfonamide;

(2-Aminomethyl-adamantan-2-yl)-methanesulfonic acid;

(1-Aminomethyl-cycloheptylmethyl)-phosphonic acid;

2-(1-Aminomethyl-cycloheptyl)-N-hydroxy-acetamide;

N-[2-(1-Aminomethyl-cycloheptyl)-ethyl]-methanesulfonamide;

3-(1-Aminomethyl-cycloheptylmethyl)-4H-[1,2,4]oxadiazole-5-thione;

N-[2-(1-Aminomethyl-cycloheptyl)-ethyl]-C,C,C-trifluoro-methanesulfonamide;

C-[1-(2-Oxo-2,3-dihydro-2,4-[1,2,3,5]oxathiadiazol-4-yl methyl)-cycloheptyl]-methylamine;

(1-Aminomethyl-cycloheptyl)-methanesulfonamide; and

(1-Aminomethyl-cycloheptyl)-methanesulfonic acid.

Another preferred embodiment of the invention method utilizes a compound of the Formula III, IIIC, IIIF, IIIG, or IIIH, wherein preferred compounds are those wherein R is a sulfonamide selected from —NHSO₂R¹⁵ or —SO₂NHR¹⁵ wherein R¹⁵ is straight or branched alkyl or trifluoromethyl.

Another preferred embodiment of the invention method utilizes a compound of the Formula III, IIIC, IIIF, IIIG, or IIIH, wherein especially preferred is N-[2-(1-aminomethyl-cyclohexyl)-ethyl]-methanesulfonamide.

Another preferred embodiment of the invention method utilizes a compound of the Formula III, IIIC, IIIF, IIIG, or IIIH, wherein other preferred compounds are those wherein R is a phosphonic acid, —PO₃H₂.

Another preferred embodiment of the invention method utilizes a compound of the Formula III, IIIC, IIIF, IIIG, or IIIH, wherein especially preferred are (1-aminomethyl-cyclohexylmethyl)-phosphonic acid and (2-aminomethyl-4-methyl-pentyl)-phosphonic acid.

Another preferred embodiment of the invention method utilizes a compound of the Formula III, IIIC, IIIF, IIIG, or IIIH, wherein other preferred compounds are those wherein R is a heterocycle selected from:

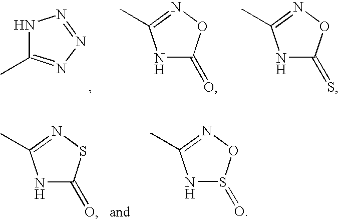

Another preferred embodiment of the invention method utilizes a compound of the Formula III, IIIC, IIIF, IIIG, or IIIH, wherein especially preferred are C-[1-(1H-tetrazol-5-ylmethyl)cyclohexyl]-methylamine and 4-methyl-2-(1H-tetrazol-5-ylmethyl)-pentylamine.

An especially preferred embodiment of the invention method utilizes a compound of the Formula III wherein:

m is an integer of from 0 to 2;

p is an integer of 2; and

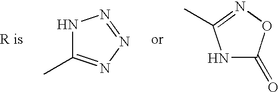

Still more preferred is an embodiment of the invention method which utilizes a compound of the Formula III, IIIC, IIIF, IIIG, or IIIH named 3-(1-aminomethyl-cyclohexylmethyl)-4H-[1,2,4]oxadiazol-5-one, or a pharmaceutically acceptable salt thereof.

Still more preferred is an embodiment of the invention method which utilizes a compound of the Formula III, IIIC, IIIF, IIIG, or IIIH named 3-(1-aminomethyl-cyclohexylmethyl)-4H-[1,2,4]oxadiazol-5-one hydrochloride.

Also preferred is an embodiment of the invention method which utilizes a compound of the Formula III, IIIC, IIIF, IIIG, or IIIH named 3-(1-aminomethyl-cycloheptylmethyl)-4H-[1,2,4]oxadiazol-5-one, or a pharmaceutically acceptable salt thereof.

Also more preferred is an embodiment of the invention method which utilizes a compound of the Formula III, IIIC, IIIF, IIIG, or IIIH named 3-(1-aminomethyl-cycloheptylmethyl)-4H-[1,2,4]oxadiazol-5-one hydrochloride.

Also preferred is an embodiment of the invention method which utilizes a compound of the Formula III, IIIC, IIIF, IIIG, or IIIH named C-[1-(1H-tetrazol-5-ylmethyl)-cycloheptyl]-methylamine, or a pharmaceutically acceptable salt thereof.

Also more preferred is an embodiment of the invention method which utilizes a compound of the Formula III, IIIC, IIIF, IIIG, or IIIH named C-[1-(1H-tetrazol-5-ylmethyl)-cycloheptyl]-methylamine.

Another preferred embodiment of the invention method utilizes an alpha2delta ligand that is a compound of the Formula IV

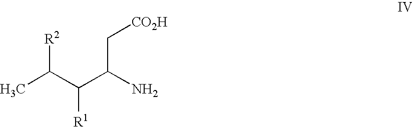

or a pharmaceutically acceptable salt thereof wherein:

R¹ is hydrogen, straight or branched alkyl of from 1 to 6 carbon atoms or phenyl;

R² is straight or branched alkyl of from 1 to 8 carbon atoms,

straight or branched alkenyl of from 2 to 8 carbon atoms,

cycloalkyl of from 3 to 7 carbon atoms,

alkoxy of from 1 to 6 carbon atoms,

-alkylcycloalkyl,

-alkylalkoxy,

-alkyl OH

-alkylphenyl,

-alkylphenoxy,

-phenyl or substituted phenyl; and

R¹ is straight or branched alkyl of from 1 to 6 carbon atoms or phenyl when R² is methyl.

Preferred is an embodiment of the invention method employing a compound of Formula IV wherein R¹ is hydrogen, and R² is alkyl.

Another preferred embodiment of the invention method employing a compound of Formula IV wherein R¹ is methyl, and R² is alkyl.

Still another preferred embodiment of the invention method utilizes a compound of Formula IV wherein R¹ is methyl, and R² is methyl or ethyl.

Especially preferred is an embodiment of the invention method utilizing a compound of Formula IV selected from:

3-Aminomethyl-5-methylheptanoic acid;

3-Aminomethyl-5-methyl-octanoic acid;

3-Aminomethyl-5-methyl-nonanoic acid;

3-Aminomethyl-5-methyl-decanoic acid;

3-Aminomethyl-5-methyl-undecanoic acid;

3-Aminomethyl-5-methyl-dodecanoic acid;

3-Aminomethyl-5-methyl-tridecanoic acid;

3-Aminomethyl-5-cyclopropyl-hexanoic acid;

3-Aminomethyl-5-cyclobutyl-hexanoic acid;

3-Aminomethyl-5-cyclopentyl-hexanoic acid;

3-Aminomethyl-5-cyclohexyl-hexanoic acid;

3-Aminomethyl-5-trifluoromethyl-hexanoic acid;

3-Aminomethyl-5-phenyl-hexanoic acid;

3-Aminomethyl-5-(2-chlorophenyl)-hexanoic acid;

3-Aminomethyl-5-(3-chlorophenyl)-hexanoic acid;

3-Aminomethyl-5-(4-chlorophenyl)-hexanoic acid;

3-Aminomethyl-5-(2-methoxyphenyl)-hexanoic acid;

3-Aminomethyl-5-(3-methoxyphenyl)-hexanoic acid;

3-Aminomethyl-5-(4-methoxyphenyl)-hexanoic acid; and

3-Aminomethyl-5-(phenylmethyl)-hexanoic acid.

Another especially preferred embodiment of the invention method uses a compound of Formula IV selected from:

(3R,4S)-3-Aminomethyl-4,5-dimethyl-hexanoic acid;

3-Aminomethyl-4,5-dimethyl-hexanoic acid;

(3R,4S)-3-Aminomethyl-4,5-dimethyl-hexanoic acid MP;

(3S,4S)-3-Aminomethyl-4,5-dimethyl-hexanoic acid;

(3R,4R)-3-Aminomethyl-4,5-dimethyl-hexanoic acid MP;

3-Aminomethyl-4-isopropyl-hexanoic acid;

3-Aminomethyl-4-isopropyl-heptanoic acid;

3-Aminomethyl-4-isopropyl-octanoic acid;

3-Aminomethyl-4-isopropyl-nonanoic acid;

3-Aminomethyl-4-isopropyl-decanoic acid; and

3-Aminomethyl-4-phenyl-5-methyl-hexanoic acid.

Another preferred embodiment of the invention method uses a compound of Formula IV selected from:

(3S,5S)-3-Aminomethyl-5-methoxy-hexanoic acid;

(3S,5S)-3-Aminomethyl-5-ethoxy-hexanoic acid;

(3S,5S)-3-Aminomethyl-5-propoxy-hexanoic acid;

(3S,5S)-3-Aminomethyl-5-isopropoxy-hexanoic acid;

(3S,5S)-3-Aminomethyl-5-tert-butoxy-hexanoic acid;

(3S,5S)-3-Aminomethyl-5-fluoromethoxy-hexanoic acid;

(3S,5S)-3-Aminomethyl-5-(2-fluoro-ethoxy)-hexanoic acid;

(3S,5S)-3-Aminomethyl-5-(3,3,3-trifluoro-propoxy)-hexanoic acid;

(3S,5S)-3-Aminomethyl-5-phenoxy-hexanoic acid;

(3S,5S)-3-Aminomethyl-5-(4-chloro-phenoxy)-hexanoic acid;

(3S,5S)-3-Aminomethyl-5-(3-chloro-phenoxy)-hexanoic acid;

(3S,5S)-3-Aminomethyl-5-(2-chloro-phenoxy)-hexanoic acid;

(3S,5S)-3-Aminomethyl-5-(4-fluoro-phenoxy)-hexanoic acid;

(3S,5S)-3-Aminomethyl-5-(3-fluoro-phenoxy)-hexanoic acid;

(3S,5S)-3-Aminomethyl-5-(2-fluoro-phenoxy)-hexanoic acid;

(3S,5S)-3-Aminomethyl-5-(4-methoxy-phenoxy)-hexanoic acid;

(3S,5S)-3-Aminomethyl-5-(3-methoxy-phenoxy)-hexanoic acid;

(3S,5S)-3-Aminomethyl-5-(2-methoxy-phenoxy)-hexanoic acid;

(3S,5S)-3-Aminomethyl-5-(4-nitro-phenoxy)-hexanoic acid;

(3S,5S)-3-Aminomethyl-5-(3-nitro-phenoxy)-hexanoic acid;

(3S,5S)-3-Aminomethyl-5-(2-nitro-phenoxy)-hexanoic acid;

(3S,5S)-3-Aminomethyl-6-hydroxy-5-methyl-hexanoic acid;

(3S,5S)-3-Aminomethyl-6-methoxy-5-methyl-hexanoic acid;

(3S,5S)-3-Aminomethyl-6-ethoxy-5-methyl-hexanoic acid;

(3S,5S)-3-Aminomethyl-5-methyl-6-propoxy-hexanoic acid;

(3S,5S)-3-Aminomethyl-6-isopropoxy-5-methyl-hexanoic acid;

(3S,5S)-3-Aminomethyl-6-tert-butoxy-5-methyl-hexanoic acid;

(3S,5S)-3-Aminomethyl-6-fluoromethoxy-5-methyl-hexanoic acid;

(3S,5S)-3-Aminomethyl-6-(2-fluoro-ethoxy)-5-methyl-hexanoic acid;

(3S,5S)-3-Aminomethyl-5-methyl-6-(3,3,3-trifluoro-propoxy)-hexanoic acid;

(3S,5S)-3-Aminomethyl-5-methyl-6-phenoxy-hexanoic acid;

(3S,5S)-3-Aminomethyl-6-(4-chloro-phenoxy)-5-methyl-hexanoic acid;

(3S,5S)-3-Aminomethyl-6-(3-chloro-phenoxy)-5-methyl-hexanoic acid;

(3S,5S)-3-Aminomethyl-6-(2-chloro-phenoxy)-5-methyl-hexanoic acid;

(3S,5S)-3-Aminomethyl-6-(4-fluoro-phenoxy)-5-methyl-hexanoic acid;

(3S,5S)-3-Aminomethyl-6-(3-fluoro-phenoxy)-5-methyl-hexanoic acid;

(3S,5S)-3-Aminomethyl-6-(2-fluoro-phenoxy)-5-methyl-hexanoic acid;

(3S,5S)-3-Aminomethyl-6-(4-methoxy-phenoxy)-5-methyl-hexanoic acid;

(3S,5S)-3-Aminomethyl-6-(3-methoxy-phenoxy)-5-methyl-hexanoic acid;

(3S,5S)-3-Aminomethyl-6-(2-methoxy-phenoxy)-5-methyl-hexanoic acid;

(3S,5S)-3-Aminomethyl-5-methyl 6-(4-trifluoromethyl-phenoxy)-hexanoic acid;

(3S,5S)-3-Aminomethyl-5-methyl 6-(3-trifluoromethyl-phenoxy)-hexanoic acid;

(3S,5S)-3-Aminomethyl-5-methyl 6-(2-trifluoromethyl-phenoxy)-hexanoic acid;

(3S,5S)-3-Aminomethyl-5-methyl 6-(4-nitro-phenoxy)-hexanoic acid;

(3S,5S)-3-Aminomethyl-5-methyl 6-(3-nitro-phenoxy)-hexanoic acid;

(3S,5S)-3-Aminomethyl-5-methyl 6-(2-nitro-phenoxy)-hexanoic acid;

(3S,5S)-3-Aminomethyl-6-benzyloxy-5-methyl-hexanoic acid;

(3S,5S)-3-Aminomethyl-7-hydroxy-5-methyl-heptanoic acid;

(3S,5S)-3-Aminomethyl-7-methoxy-5-methyl-heptanoic acid;

(3S,5S)-3-Aminomethyl-7-ethoxy-5-methyl-heptanoic acid;

(3S,5S)-3-Aminomethyl-5-methyl-7-propoxy-heptanoic acid;

(3S,5S)-3-Aminomethyl-7-isopropoxy-5-methyl-heptanoic acid;

(3S,5S)-3-Aminomethyl-7-tert-butoxy-5-methyl-heptanoic acid;

(3S,5S)-3-Aminomethyl-7-fluoromethoxy-5-methyl-heptanoic acid;

(3S,5S)-3-Aminomethyl-7-(2-fluoro-ethoxy)-5-methyl-heptanoic acid;

(3S,5S)-3-Aminomethyl-5-methyl-7-(3,3,3-trifluoro-propoxy)-heptanoic acid;

(3S,5S)-3-Aminomethyl-7-benzyloxy-5-methyl-heptanoic acid;

(3S,5S)-3-Aminomethyl-5-methyl-7-phenoxy-heptanoic acid;

(3S,5S)-3-Aminomethyl-7-(4-chloro-phenoxy)-5-methyl-heptanoic acid;

(3S,5S)-3-Aminomethyl-7-(3-chloro-phenoxy)-5-methyl-heptanoic acid;

(3S,5S)-3-Aminomethyl-7-(2-chloro-phenoxy)-5-methyl-heptanoic acid;

(3S,5S)-3-Aminomethyl-7-(4-fluoro-phenoxy)-5-methyl-heptanoic acid;

(3S,5S)-3-Aminomethyl-7-(3-fluoro-phenoxy)-5-methyl-heptanoic acid;

(3S,5S)-3-Aminomethyl-7-(2-fluoro-phenoxy)-5-methyl-heptanoic acid;

(3S,5S)-3-Aminomethyl-7-(4-methoxy-phenoxy)-5-methyl-heptanoic acid;

(3S,5S)-3-Aminomethyl-7-(3-methoxy-phenoxy)-5-methyl-heptanoic acid;

(3S,5S)-3-Aminomethyl-7-(2-methoxy-phenoxy)-5-methyl-heptanoic acid;

(3S,5S)-3-Aminomethyl-5-methyl-7-(4-trifluoromethyl-phenoxy)-heptanoic acid;

(3S,5S)-3-Aminomethyl-5-methyl-7-(3-trifluoromethyl-phenoxy)-heptanoic acid;

(3S,5S)-3-Aminomethyl-5-methyl-7-(2-trifluoromethyl-phenoxy)-heptanoic acid;

(3S,5S)-3-Aminomethyl-5-methyl-7-(4-nitro-phenoxy)-heptanoic acid;

(3S,5S)-3-Aminomethyl-5-methyl-7-(3-nitro-phenoxy)-heptanoic acid;

(3S,5S)-3-Aminomethyl-5-methyl-7-(2-nitro-phenoxy)-heptanoic acid;

(3S,5S)-3-Aminomethyl-5-methyl-6-phenyl-hexanoic acid;

(3S,5S)-3-Aminomethyl-6-(4-chloro-phenyl)-5-methyl-hexanoic acid;

(3S,5S)-3-Aminomethyl-6-(3-chloro-phenyl)-5-methyl-hexanoic acid;

(3S,5S)-3-Aminomethyl-6-(2-chloro-phenyl)-5-methyl-hexanoic acid;

(3S,5S)-3-Aminomethyl-6-(4-methoxy-phenyl)-5-methyl-hexanoic acid;

(3S,5S)-3-Aminomethyl-6-(3-methoxy-phenyl)-5-methyl-hexanoic acid;

(3S,5S)-3-Aminomethyl-6-(2-methoxy-phenyl)-5-methyl-hexanoic acid;

(3S,5S)-3-Aminomethyl-6-(4-fluoro-phenyl)-5-methyl-hexanoic acid;

(3S,5S)-3-Aminomethyl-6-(3-fluoro-phenyl)-5-methyl-hexanoic acid;

(3S,5S)-3-Aminomethyl-6-(2-fluoro-phenyl)-5-methyl-hexanoic acid;

(3S,5R)-3-Aminomethyl-5-methyl-7-phenyl-heptanoic acid;

(3S,5R)-3-Aminomethyl-7-(4-chloro-phenyl)-5-methyl-heptanoic acid;

(3S,5R)-3-Aminomethyl-7-(3-chloro-phenyl)-5-methyl-heptanoic acid;

(3S,5R)-3-Aminomethyl-7-(2-chloro-phenyl)-5-methyl-heptanoic acid;

(3S,5R)-3-Aminomethyl-7-(4-methoxy-phenyl)-5-methyl-heptanoic acid;

(3S,5R)-3-Aminomethyl-7-(3-methoxy-phenyl)-5-methyl-heptanoic acid;

(3S,5R)-3-Aminomethyl-7-(2-methoxy-phenyl)-5-methyl-heptanoic acid;

(3S,5R)-3-Aminomethyl-7-(4-fluoro-phenyl)-5-methyl-heptanoic acid;

(3S,5R)-3-Aminomethyl-7-(3-fluoro-phenyl)-5-methyl-heptanoic acid;

(3S,5R)-3-Aminomethyl-7-(2-fluoro-phenyl)-5-methyl-heptanoic acid;

(3S,5R)-3-Aminomethyl-5-methyl-oct-7-enoic acid;

(3S,5R)-3-Aminomethyl-5-methyl-non-8-enoic acid;

(E)-(3S,5S)-3-Aminomethyl-5-methyl-oct-6-enoic acid;

(Z)-(3S,5S)-3-Aminomethyl-5-methyl-oct-6-enoic acid;

(Z)-(3S,5S)-3-Aminomethyl-5-methyl-non-6-enoic acid;

(E)-(3S,5S)-3-Aminomethyl-5-methyl-non-6-enoic acid;

(E)-(3S,5R)-3-Aminomethyl-5-methyl-non-7-enoic acid;

(Z)-(3S,5R)-3-Aminomethyl-5-methyl-non-7-enoic acid;

(Z)-(3S,5R)-3-Aminomethyl-5-methyl-dec-7-enoic acid;

(E)-(3S,5R)-3-Aminomethyl-5-methyl-undec-7-enoic acid;

(3S,5S)-3-Aminomethyl-5,6,6-trimethyl-heptanoic acid;

(3S,5S)-3-Aminomethyl-5,6-dimethyl-heptanoic acid;

(3S,5S)-3-Aminomethyl-5-cyclopropyl-hexanoic acid;

(3S,5S)-3-Aminomethyl-5-cyclobutyl-hexanoic acid;

(3S,5S)-3-Aminomethyl-5-cyclopentyl-hexanoic acid; and

(3S,5S)-3-Aminomethyl-5-cyclohexyl-hexanoic acid.

Still another more preferred embodiment of the invention method utilizes a compound of Formula IV selected from:

(3S,5R)-3-Aminomethyl-5-methyl-heptanoic acid;

(3S,5R)-3-Aminomethyl-5-methyl-octanoic acid;

(3S,5R)-3-Aminomethyl-5-methyl-nonanoic acid;

(3S,5R)-3-Aminomethyl-5-methyl-decanoic acid;

(3S,5R)-3-Aminomethyl-5-methyl-undecanoic acid;

(3S,5R)-3-Aminomethyl-5-methyl-dodecanoic acid;

(3S,5R)-3-Aminomethyl-5,9-dimethyl-decanoic acid;

(3S,5R)-3-Aminomethyl-5,7-dimethyl-octanoic acid;

(3S,5R)-3-Aminomethyl-5,8-dimethyl-nonanoic acid;

(3S,5R)-3-Aminomethyl-6-cyclopropyl-5-methyl-hexanoic acid;

(3S,5R)-3-Aminomethyl-6-cyclobutyl-5-methyl-hexanoic acid;

(3S,5R)-3-Aminomethyl-6-cyclopentyl-5-methyl-hexanoic acid;

(3S,5R)-3-Aminomethyl-6-cyclohexyl-5-methyl-hexanoic acid;

(3S,5R)-3-Aminomethyl-7-cyclopropyl-5-methyl-heptanoic acid;

(3S,5R)-3-Aminomethyl-7-cyclobutyl-5-methyl-heptanoic acid;

(3S,5R)-3-Aminomethyl-7-cyclopentyl-5-methyl-heptanoic acid;

(3S,5R)-3-Aminomethyl-7-cyclohexyl-5-methyl-heptanoic acid;

(3S,5R)-3-Aminomethyl-8-cyclopropyl-5-methyl-octanoic acid;

(3S,5R)-3-Aminomethyl-8-cyclobutyl-5-methyl-octanoic acid;

(3S,5R)-3-Aminomethyl-8-cyclopentyl-5-methyl-octanoic acid;

(3S,5R)-3-Aminomethyl-8-cyclohexyl-5-methyl-octanoic acid;

(3S,5S)-3-Aminomethyl-6-fluoro-5-methyl-hexanoic acid;

(3S,5S)-3-Aminomethyl-7-fluoro-5-methyl-heptanoic acid;

(3S,5R)-3-Aminomethyl-8-fluoro-5-methyl-octanoic acid;

(3S,5R)-3-Aminomethyl-9-fluoro-5-methyl-nonanoic acid;

(3S,5S)-3-Aminomethyl-7,7,7-trifluoro-5-methyl-heptanoic acid;

(3S,5R)-3-Aminomethyl-8,8,8-trifluoro-5-methyl-octanoic acid;

(3S,5R)-3-Aminomethyl-5-methyl-8-phenyl-octanoic acid;

(3S,5S)-3-Aminomethyl-5-methyl-6-phenyl-hexanoic acid; and

(3S,5R)-3-Aminomethyl-5-methyl-7-phenyl-heptanoic acid.

Another preferred embodiment of the invention method utilizes an alpha2delta ligand which is a compound of the Formula (IXA) or (IXB)

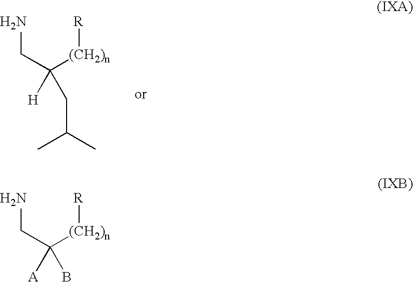

or a pharmaceutically acceptable salt thereof wherein:

n is an integer of from 0 to 2;

R is sulfonamide,

amide,

phosphonic acid,

heterocycle,

sulfonic acid, or

hydroxamic acid;

A is hydrogen or methyl; and

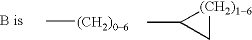

straight or branched alkyl of from 1 to 11 carbons, or

—(CH₂)₁₋₄₋Y—(CH₂)₀₋₄₋phenyl wherein Y is —O—, —S—, —NR′₃ wherein:

R′₃ is alkyl of from 1 to 6 carbons, cycloalkyl of from 3 to 8 carbons, benzyl or phenyl wherein benzyl or phenyl can be unsubstituted or substituted with from 1 to 3 substituents each independently selected from alkyl, alkoxy, halogen, hydroxy, carboxy, carboalkoxy, trifluoromethyl, and nitro.

A more preferred embodiment of the invention method utilizes an alpha2delta ligand which is a compound of the Formula (IXA) or (IXB), wherein R is a sulfonamide selected from —NHSO₂R¹⁵ and —SO₂NHR¹⁵, wherein R¹⁵ is straight or branched alkyl or trifluoromethyl.

An especially preferred embodiment of the invention method utilizes a compound of the Formula (IXA) or (IXB) selected from:

4-Methyl-2-(1H-tetrazol-5-yl methyl)-pentylamine;

3-(2-Aminomethyl-4-methyl-pentyl)-4H-[1,2,4]oxadiazole-5-thione, HCl;

(2-Aminomethyl-4-methyl-pentyl)-phosphonic acid;

3-(3-Amino-2-cyclopentyl-propyl)-4H-[1,2,4]oxadiazol-5-one;

3-(3-Amino-2-cyclopentyl-propyl)-4H-[1,2,4]thiadiazol-5-one;

2-Cyclopentyl-3-(2-oxo-2,3-dihydro-2λ⁴-[1,2,3,5]oxathiadiazol-4-yl)-propylamine;

3-(3-Amino-2-cyclobutyl-propyl)-4H-[1,2,4]oxadiazol-5-one;

3-(3-Amino-2-cyclobutyl-propyl)-4H-[1,2,4]thiadiazol-5-one; and

2-Cyclobutyl-3-(2-oxo-2,3-dihydro-2λ⁴-[1,2,3,5]oxathiadiazol-4-yl)-propylamine.

Another preferred embodiment of the invention method utilizes a compound of the Formula (IXA) or (IXB), wherein R is a phosphonic acid, —PO₃H₂.

Another preferred embodiment of the invention method utilizes a compound of the Formula (IXA) or (IXB), wherein R is

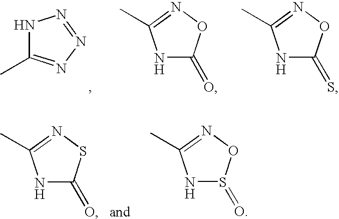

More preferred is an embodiment of the invention method that utilizes a compound of the Formula (IXA) or (IXB), wherein R is

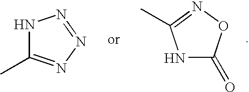

Still more preferred is an embodiment of the invention method that utilizes a compound of the Formula (IXA) or (IXB) that is 3-(2-aminomethyl-4-methyl-pentyl)-4H-[1,3,4]oxadiazol-5-one, or a pharmaceutically acceptable salt thereof.

Still more preferred is an embodiment of the invention method that utilizes a compound of the Formula (IXA) or (IXB) that is 3-(2-aminomethyl-4-methyl-pentyl)-4H-[1,2,4]oxadiazol-5-one hydrochloride.

Another embodiment utilizes an alpha2delta ligand that is a compound of the Formula V, VI, VII, or VIII

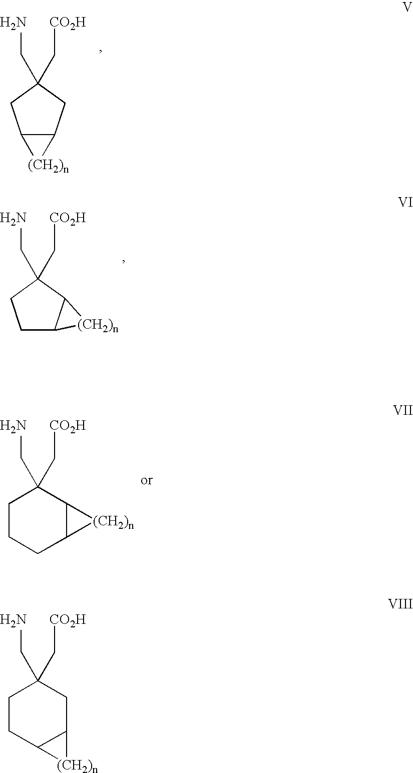

or a pharmaceutically acceptable salt thereof, wherein n is integer of from 1 to 4, where there are stereocenters, each center may be independently R or S.

A preferred embodiment of the invention method utilizes a compound of the Formula V, VI, VII, or VIII, wherein n is an integer of from 2 to 4.

Another preferred embodiment of the invention method utilizes a compound of the Formula V.

A still more preferred embodiment of the invention method utilizes a compound of the Formula V, VI, VII, or VIII that is selected from:

(1α,6α,8β)(2-Aminomethyl-octahydro-inden-2-yl)-acetic acid;

(2-Aminomethyl-octahydro-inden-2-yl)-acetic acid;

(2-Aminomethyl-octahydro-pentalen-2-yl)-acetic acid;

(2-Aminomethyl-octahydro-pentalen-2-yl)-acetic acid;

(3-Aminomethyl-bicyclo[3.2.0]hept-3-yl)-acetic acid;

(3-Aminomethyl-bicyclo[3.2.0]hept-3-yl)-acetic acid; and

(2-Aminomethyl-octahydro-inden-2-yl)-acetic acid;

Another still more preferred embodiment of the invention method utilizes a compound of the Formula V, VI, VII, or VIII that is selected from:

(1α,5β)(3-Aminomethyl-bicyclo[3.1.0]hex-3-yl)-acetic acid,

(1α,5β)(3-Aminomethyl-bicyclo[3.2.0]hept-3-yl)-acetic acid,

(1α,5β)(2-Aminomethyl-octahydro-pentalen-2-yl)-acetic acid,

(1α,6β)(2-Aminomethyl-octahydro-inden-2-yl)-acetic acid,

(1α,7β)(2-Aminomethyl-decahydro-azulen-2-yl)-acetic acid,

(1α,5β)(3-Aminomethyl-bicyclo[3.1.0]hex-3-yl)-acetic acid,

(1α,5β)(3-Aminomethyl-bicyclo[3.2.0]hept-3-yl)-acetic acid,

(1α,5β)(2-Aminomethyl-octahydro-pentalen-2-yl)-acetic acid,

(1α,6β)(2-Aminomethyl-octahydro-inden-2-yl)-acetic acid,

(1α,7β)(2-Aminomethyl-decahydro-azulen-2-yl)-acetic acid,

(1α,3α,5α)(3-Aminomethyl-bicyclo[3.1.0]hex-3-yl)-acetic acid,

(1α,3α,5α)(2-Aminomethyl-octahydro-pentalen-2-yl)-acetic acid,

(1α,6α,8α)(2-Aminomethyl-octahydro-inden-2-yl)-acetic acid,

(1α,7α,9α)(2-Aminomethyl-decahydro-azulen-2-yl)-acetic acid,

(1α,3β,5α)(3-Aminomethyl-bicyclo[3.1.0]hex-3-yl)-acetic acid,

(1α,3β,5α)(3-Aminomethyl-bicyclo[3.2.0]hept-3-yl)-acetic acid,

(1α,3β,5α)(2-Aminomethyl-octahydro-pentalen-2-yl)-acetic acid,

(1α,6α,8β)(2-Aminomethyl-octahydro-inden-2-yl)-acetic acid,

(1α,7α,9β)(2-Aminomethyl-decahydro-azulen-2-yl)-acetic acid,

((1R,3R,6R)-3-Aminomethyl-bicyclo[4.1.0]hept-3-yl)-acetic acid,

((1R,3S,6R)-3-Aminomethyl-bicyclo[4.1.0]hept-3-yl)-acetic acid,

((1S,3S,6S)-3-Aminomethyl-bicyclo[4.1.0]hept-3-yl)-acetic acid,

((1S,3R,6S)-3-Aminomethyl-bicyclo[4.1.0]hept-3-yl)-acetic acid,

((1R,3R,6S)-3-Aminomethyl-bicyclo[4.2.0]oct-3-yl)-acetic acid,

((1R,3S,6S)-3-Aminomethyl-bicyclo[4.2.0]oct-3-yl)-acetic acid,

((1S,3S,6R)-3-Aminomethyl-bicyclo[4.2.0]oct-3-yl)-acetic acid,

((1S,3R,6R)-3-Aminomethyl-bicyclo[4.2.0]oct-3-yl)-acetic acid,

((3αR,5R,7αS)-5-Aminomethyl-octahydro-inden-5-yl)-acetic acid,

((3αR,5S,7αS)-5-Aminomethyl-octahydro-inden-5-yl)-acetic acid,

((3αS,5S,7αR)-5-Aminomethyl-octahydro-inden-5-yl)-acetic acid,

((3αS,5R,7αR)-5-Aminomethyl-octahydro-inden-5-yl)-acetic acid,

((2R,4αS,8αR)-2-Aminomethyl-decahydro-naphthalen-2-yl)-acetic acid,

((2S,4αS,8αR)-2-Aminomethyl-decahydro-naphthalen-2-yl)-acetic acid,

((2S,4αR,8αS)-2-Aminomethyl-decahydro-naphthalen-2-yl)-acetic acid,

((2R,4αR,8αS)-2-Aminomethyl-decahydro-naphthalen-2-yl)-acetic acid,

((2R,4αS,9αR)-2-Aminomethyl-decahydro-benzocyclophepten-2-yl)-acetic acid,

((2S,4αS,9αR)-2-Aminomethyl-decahydro-benzocyclophepten-2-yl)-acetic acid,

((2S,4αR,9αS)-2-Aminomethyl-decahydro-benzocyclophepten-2-yl)-acetic acid,

((2R,4αR,9αS)-2-Aminomethyl-decahydro-benzocyclophepten-2-yl)-acetic acid,

((1R,3R,6S)-3-Aminomethyl-bicyclo[4.1.0]hept-3-yl)-acetic acid,

((1R,3S,6S)-3-Aminomethyl-bicyclo[4.1.0]hept-3-yl)-acetic acid,

((1S,3S,6R)-3-Aminomethyl-bicyclo[4.1.0]hept-3-yl)-acetic acid,

((1S,3R,6R)-3-Aminomethyl-bicyclo[4.1.0]hept-3-yl)-acetic acid,

((1R,3R,6R)-3-Aminomethyl-bicyclo[4.2.0]oct-3-yl)-acetic acid,

((1R,3S,6R)-3-Aminomethyl-bicyclo[4.2.0]oct-3-yl)-acetic acid,

((1S,3S,6S)-3-Aminomethyl-bicyclo[4.2.0]oct-3-yl)-acetic acid,

((1S,3R,6S)-3-Aminomethyl-bicyclo[4.2.0]oct-3-yl)-acetic acid,

((3αR,5R,7αR)-5-Aminomethyl-octahydro-inden-5-yl)-acetic acid,

((3αR,5S,7αR)-5-Aminomethyl-octahydro-inden-5-yl)-acetic acid,

((3αS,5S,7αS)-5-Aminomethyl-octahydro-inden-5-yl)-acetic acid,

((3αS,5R,7αS)-5-Aminomethyl-octahydro-inden-5-yl)-acetic acid,

((2R,4αR,8αR)-2-Aminomethyl-decahydro-naphthalen-2-yl)-acetic acid,

((2S,4αS,8αR)-2-Aminomethyl-decahydro-naphthalen-2-yl)-acetic acid,

((2S,4αR,8αS)-2-Aminomethyl-decahydro-naphthalen-2-yl)-acetic acid,

((2R,4αS,8αS)-2-Aminomethyl-decahydro-naphthalen-2-yl)-acetic acid,

((2R,4αR,9αR)-2-Aminomethyl-decahydro-benzocyclophepten-2-yl)-acetic acid,

((2S,4αR,9αR)-2-Aminomethyl-decahydro-benzocyclophepten-2-yl)-acetic acid,

((2S,4αS,9αS)-2-Aminomethyl-decahydro-benzocyclophepten-2-yl)-acetic acid, and

((2R,4αS,9αS)-2-Aminomethyl-decahydro-benzocyclophepten-2-yl)-acetic acid.

A more preferred embodiment of the invention method utilizes an alpha2delta ligand of the Formula V, VI, VII, or VIII that is (1α,3α,5α)(3-aminomethyl-bicyclo[3.2.0]hept-3-yl)-acetic acid, or a pharmaceutically acceptable salt thereof.

A still more preferred embodiment of the invention method utilizes an alpha2delta ligand of the Formula V, VI, VII, or VIII that is (1α,3α,5α)(3-aminomethyl-bicyclo[3.2.0]hept-3-yl)-acetic acid hydrochloride.

Other preferred embodiments of the invention method are those wherein the alpha2delta ligand that is employed is selected from the following compounds and their pharmaceutically acceptable salts:

3-(1-aminomethyl-cyclohexylmethyl)-4H-[1,2,4]oxadiazol-5-one;

(S,S)-(1-aminomethyl-3,4-dimethyl-cyclopentyl)-acetic acid;

(R,S)-3-aminomethyl-5-methyl-octanoic acid;

(S,R)-3-aminomethyl-5-methyl-octanoic acid;

(3-aminomethyl-bicyclo[3.2.0]hept-3-yl)-acetic acid;

(3-aminomethyl-bicyclo[3.2.0]hept-3-yl)-acetic acid, wherein the cyclobutyl ring is trans to the methylamine group; and

C-[1-(H-tetrazol-5-ylmethyl)-cycloheptyl]-methylamine.

These compounds can be prepared as described below or in PCT Patent Application WO 99/21824, published May 6, 1999, PCT Patent Application WO 00/76958, published Dec. 21, 2000, or PCT Patent Application WO 01/28978, published Apr. 26, 2001. These applications are incorporated herein by reference in their entireties.

A more preferred embodiment of the invention method utilizes the hydrochloride salt of the compound 3-(1-aminomethyl-cyclohexylmethyl)-4H-[1,2,4]oxadiazol-5-one.

Another preferred embodiment of the invention method utilizes the cyclic amino acids of the Formula I. These are described in U.S. Pat. No. 4,024,175 and U.S. Pat. No. 4,087,544, which are both incorporated herein by reference in their entireties.

Another preferred embodiment of the invention method utilizes an alpha2delta ligand of the Formula II, and these compounds are described in U.S. Pat. No. 5,563,175, which is incorporated herein by reference in its entirety.

Another preferred embodiment of the invention method utilizes an alpha2delta ligand of the Formula III, IIIC, IIIF, IIIG, or IIIH. These compounds are described in PCT Patent Application No. WO 99/31075, which is incorporated herein by reference in its entirety.

Another preferred embodiment of the invention method utilizes an alpha2delta ligand of the Formula IV, which are described in PCT Patent Application No. WO 00/76958, which is incorporated herein by reference in its entirety.

Other preferred alpha2delta ligands to be utilized in the invention method are compounds of the Formula (IXA) and (IXB), which are described in PCT Patent Application No. WO 99/31074, which is incorporated herein by reference in its entirety.

PCT Patent Application No. WO 01/28978, which is incorporated herein by reference in its entirety, describes other preferred alpha2delta ligands that can be utilized in preferred embodiments of the invention. Such compounds are compounds of the Formulas V, VI, VII, and VIII.

Other alpha2delta ligands that can be used in preferred embodiments of the present invention method are described in PCT Patent Application No. WO 99/31057, which is incorporated herein by reference in its entirety. Such alpha2delta ligands are compounds of the Formulas (XII) and (XIII)

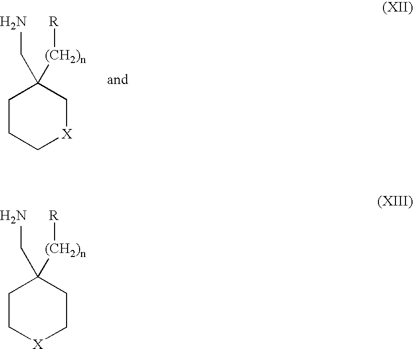

or a pharmaceutically acceptable salt thereof wherein:

n is an integer of from 0 to 2;

R is sulfonamide,

amide,

phosphonic acid,

heterocycle,

sulfonic acid, or

hydroxamic acid; and

X is —O—, —S—, —S(O)—, —S(O)₂—, or NR′₁ wherein R′₁ is hydrogen, straight or branched alkyl of from 1 to 6 carbons, benzyl, —C(O)R′₂ wherein R′₂ is straight or branched alkyl of 1 to 6 carbons, benzyl or phenyl or —CO₂R′₃ wherein R′₃ is straight or branched alkyl of from 1 to 6 carbons, or benzyl wherein the benzyl or phenyl groups can be unsubstituted or substituted by from 1 to 3 substituents selected from halogen, trifluoromethyl, and nitro.

Other alpha2delta ligands that may be utilized in preferred embodiments of the invention method are described in PCT Patent Application No. WO 98/17627, which is incorporated herein by reference in its entirety. Such alpha2delta ligands are compounds of the formula

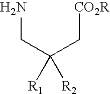

or a pharmaceutically acceptable salt thereof wherein:

R is hydrogen or lower alkyl;

R₁ is hydrogen or lower alkyl;

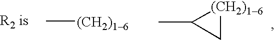

straight or branched alkyl of from 7 to 11 carbon atoms, or

—(CH₂)₍₁₋₄₎—X—(CH₂)₍₀₋₄₎-phenyl wherein

X is —O—, —S—, —NR₃₋ wherein

R₃ is alkyl of from 1 to 6 carbons, cycloalkyl of from 3 to 8 carbons, benzyl or phenyl;

wherein phenyl and benzyl can be unsubstituted or substituted with from 1 to 3 substituents each independently selected from alkyl, alkoxy, halogen, hydroxy, carboxy, carboalkoxy, trifluoromethyl, amino, and nitro.

Other alpha2delta ligands that can be utilized in preferred embodiments of the invention method are described in PCT Patent Application No. WO 99/61424, which is incorporated herein by reference in its entirety. Such alpha2delta ligands are compounds of the formulas (1), (2), (3), (4), (5), (6), (7), and (8)

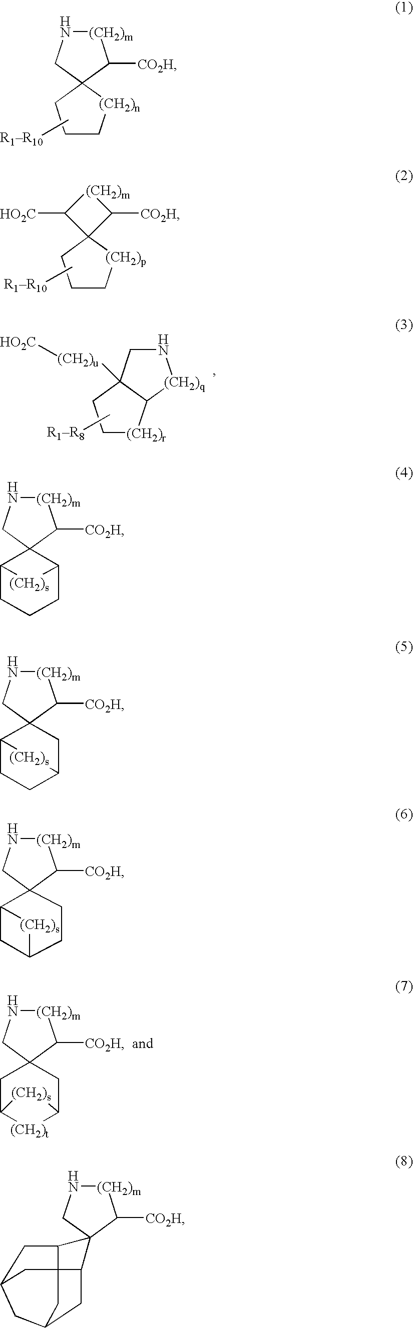

and the pharmaceutically acceptable salts and prodrugs of such compounds wherein:

R₁ to R₁₀ are each independently selected from hydrogen or a straight or branched alkyl of from 1 to 6 carbons, benzyl, or phenyl;

m is an integer of from 0 to 3;

n is an integer of from 1 to 2;

o is an integer of from 0 to 3;

p is an integer of from 1 to 2;

q is an integer of from 0 to 2;

r is an integer of from 1 to 2;

s is an integer of from 1 to 3;

t is an integer of from 0 to 2; and

u is an integer of from 0 to 1.

Other alpha2delta ligands that can be utilized in preferred embodiments of the invention method are described in U.S. Provisional Patent Application No. 60/353,632, filed on Jan. 31, 2002. Such alpha2delta ligands are compounds of the formulas X, XA, XB, XI, XIA, XIB and XB-1, as described below. Compounds of the formula X have the formula

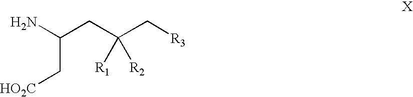

wherein

R₁ is hydrogen or (C₁-C₃)alkyl optionally substituted with from one to five fluorine atoms;

R₂ is hydrogen or (C₁-C₃)alkyl optionally substituted with from one to five fluorine atoms;

R₃ is (C₁-C₆)alkyl, (C₃-C₆)cycloalkyl, (C₃-C₆)cycloalkyl-(C₁-C₃)alkyl, phenyl, phenyl-(C₁-C₃)alkyl, pyridyl, pyridyl-(C₁-C₃)alkyl, phenyl-N(H)—, or pyridyl-N(H)—, wherein each of the foregoing alkyl moieties can be optionally substituted with from one to five fluorine atoms, preferably with from zero to three fluorine atoms, and wherein said phenyl and said pyridyl and the phenyl and pyridyl moieties of said phenyl-(C₁-C₃)alkyl and said pyridyl-(C₁-C₃)alkyl, respectively, can be optionally substituted with from one to three substituents, preferably with from zero to two substituents, independently selected from chloro, fluoro, amino, nitro, cyano, (C₁-C₃)alkylamino, (C₁-C₃)alkyl optionally substituted with from one to three fluorine atoms and (C₁-C₃)alkoxy optionally substituted with from one to three fluorine atoms;

with the proviso that when R₁ is hydrogen, R₂ is not hydrogen;

and the pharmaceutically acceptable salts of such compounds.

Compounds of the formula XI have the formula

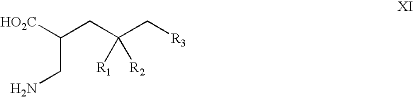

wherein R₁, R₂, and R₃ are defined as above, and the pharmaceutically acceptable salts of such compounds.

Compounds of the formula XA have the formula

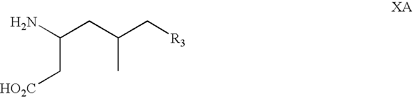

wherein R₃ is defined as above, and the pharmaceutically acceptable salts of such compounds.

Compounds of the formula XIA have the formula

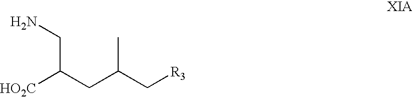

wherein R₃ is defined as above, and the pharmaceutically acceptable salts of such compounds.

Compounds of the formula XIB have the formula

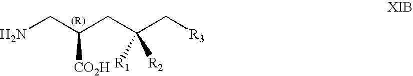

wherein R₁, R₂, and R₃ are defined as above.

Compounds of the formula XB have the formula

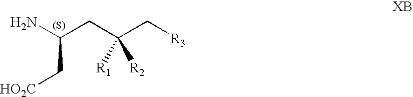

wherein R₁, R₂, and R₃ are defined as above.

Compounds of the formula XB-I have the formula

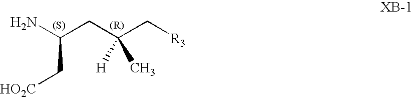

wherein R₃ is defined as above.

All U.S. patents and WO publications referenced above are incorporated herein by reference in their entireties.

It should be appreciated that the terms “uses”, “utilizes”, and “employs” are used interchangeably when describing an embodiment of the present invention.

The phrase “lower alkyl” means a straight or branched alkyl group or radical having from 1 to 6 carbon atoms, and includes methyl, ethyl, n-propyl, i-propyl, n-butyl, i-butyl, sec-butyl, tert-butyl, n-pentyl, n-hexyl, and the like.

The term “alkyl” is a straight or branched group of from 1 to 8 carbon atoms, unless stated otherwise, including but not limited to methyl, ethyl, propyl, n-propyl, isopropyl, butyl, 2-butyl, tert-butyl, and octyl. Alkyl can be unsubstituted or substituted by hydroxy or from 1 to 3 fluorine atoms. Preferred groups are methyl and ethyl.

The term “alkenyl” is a straight or branched group of from 2 to 8 carbon atoms containing 1 or 2 or 3 double bonds including but not limited to ethenyl, propen-1-yl, propen-2-yl, propen-3-yl, 1-hexen-3-yl, and hept-1,3-dien-7-yl. Alkenyl can be unsubstituted or substituted by from 1 to 3 fluorine atoms.

The term “cycloalkyl” means a cyclic group of from 3 to 7 carbon atoms including but not limited to cyclopropyl, cyclobutyl, and cycloheptyl.

The benzyl and phenyl groups may be unsubstituted or substituted with from 1 to 3 groups each independently selected from halogen, especially fluoro, alkoxy, alkyl, and NH₂.

“Halogen” includes fluorine, chlorine, bromine, and iodine.

The term “alkoxy” means the group —O-alkyl wherein alkyl is as defined above.

Sulfonamides are those of formula —NHSO₂R¹⁵ or —SO₂NHR¹⁵ wherein R¹⁵ is a straight or branched alkyl group of from 1 to 6 carbons or a trifluoromethyl.

Amides are compounds of formula —NHCOR¹² wherein R¹² is straight or branched alkyl of from 1 to 6 carbons, benzyl, and phenyl.

Phosphonic acids are —PO₃H₂.

Sulfonic acids are —SO₃H.

Hydroxamic acid is

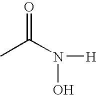

Heterocycles are groups of from 1 to 2 rings, the monocyclic rings having from 4 to 7 ring members and the bicyclic ring having from 7 to 12 ring members, with from 1 to 6 heteroatoms selected from oxygen, nitrogen, and sulfur.

Preferred heterocycles are

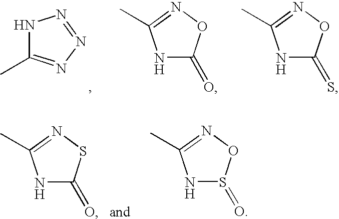

The term alkyl is a straight or branched group of from 1 to 11 carbon atoms including but not limited to methyl, ethyl, propyl, n-propyl, isopropyl, butyl, 2-butyl, tert-butyl, pentyl, hexyl, and n-hexyl, heptyl, octyl, nonyl, decyl, and undecyl except as where otherwise stated.

The cycloalkyl groups are from 3 to 8 carbons and are cyclopropyl, cyclobutyl, cyclopentyl, cyclohexyl, cycloheptyl, and cyclooctyl unless otherwise stated.

The benzyl and phenyl groups may be unsubstituted or substituted by from 1 to 3 substituents selected from hydroxy, carboxy, carboalkoxy, halogen, CF₃, nitro, alkyl, and alkoxy. Preferred are fluorine and chlorine.

Carboalkoxy is —COOalkyl wherein alkyl is as described above. Preferred are carbomethoxy and carboethoxy.

### DETAILED DESCRIPTION OF THE INVENTION

The degree of binding to the α2δ subunit can be determined using the radioligand binding assay using [3H]gabapentin and the α2β subunit derived from porcine brain tissue, as described by N. S. Gee et al., J. Biol. Chem., 1996, 271:5879-5776.

The ability of a compound to treat ADHD can be evaluated using the method described by Carol A. Bauer in “Assessing ADHD and Prospective ADHD Therapeutics Using a Psychological Animal Model”, Journal of the Association for Research in Otolaryngology, 2/1:054-064 (2001).

All that is required to practice the method of this invention is to administer an alpha2delta ligand, or a pharmaceutically acceptable salt thereof, in an amount that is therapeutically effective to treat ADHD. Such ADHD-treating amount will generally be from about 1 to about 300 mg/kg of subject body weight. Typical doses will be from about 10 to about 5000 mg/day for an adult subject of normal weight. In a clinical setting, regulatory agencies such as, for example, the Food and Drug Administration (“FDA”) in the U.S. may require a particular therapeutically effective amount.

In determining what constitutes an effective amount or a therapeutically effective amount of an alpha2delta ligand, or a pharmaceutically acceptable salt thereof, for treating ADHD according to the invention method, a number of factors will generally be considered by the medical practitioner or veterinarian in view of the experience of the medical practitioner or veterinarian, published clinical studies, the subject's (ie, mammal's) age, sex, weight and general condition, as well as the type and extent of the disease, disorder or condition being treated, and the use of other medications, if any, by the subject. As such, the administered dose may fall within the ranges or concentrations recited above, or may vary outside, i.e., either below or above, those ranges depending upon the requirements of the individual subject, the severity of the condition being treated, and the particular therapeutic formulation being employed. Determination of a proper dose for a particular situation is within the skill of the medical or veterinary arts. Generally, treatment may be initiated using smaller dosages of the alpha2delta ligand that are less than optimum for a particular subject. Thereafter, the dosage can be increased by small increments until the optimum effect under the circumstance is reached. For convenience, the total daily dosage may be divided and administered in portions during the day, if desired.

Pharmaceutical compositions of an alpha2delta ligand, or a pharmaceutically acceptable salt thereof, are produced by formulating the active compound in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the invention can also contain other components such as coloring agents, flavoring agents, and/or preservatives. These materials, if present, are usually used in relatively small amounts. The compositions can, if desired, also contain other therapeutic agents commonly employed to treat ADHD. Further, the compositions can, if desired, also contain other therapeutic agents commonly employed to treat secondary symptoms such as, for example, depression or anxiety that may or may not accompany ADHD. For example, the compositions may contain sertraline, fluoxetine, or other antidepressant or antianxiety agents.

The percentage of the active ingredients in the foregoing compositions can be varied within wide limits, but for practical purposes it is preferably present in a concentration of at least 10% in a solid composition and at least 2% in a primary liquid composition. The most satisfactory compositions are those in which a much higher proportion of the active ingredient is present, for example, up to about 95%.

Preferred routes of administration of an alpha2delta ligand, or a pharmaceutically acceptable salt thereof, are oral or parenteral. For example, a useful intravenous dose is between 5 and 50 mg, and a useful oral dosage is between 20 and 800 mg.

The alpha2delta ligand, or a pharmaceutically acceptable salt thereof, may be administered in any form. Preferably, administration is in unit dosage form. A unit dosage form of the alpha2delta ligand, or a pharmaceutically acceptable salt thereof, to be used in this invention may also comprise other compounds useful in the therapy of diseases resulting in ADHD.

The invention method is useful in human and veterinary medicines for treating or preventing ADHD in a mammal.

Some of the compounds utilized in a method of the present invention are capable of further forming pharmaceutically acceptable salts, including, but not limited to, acid addition and/or base salts. The acid addition salts are formed from basic compounds, whereas the base addition salts are formed from acidic compounds. All of these forms are within the scope of the compounds useful in the method of the present invention.

Pharmaceutically acceptable acid addition salts of the basic compounds useful in the method of the present invention include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977;66:1).

An acid addition salt of a basic compound useful in the method of the present invention is prepared by contacting the free base form of the compound with a sufficient amount of a desired acid to produce a nontoxic salt in the conventional manner. The free base form of the compound may be regenerated by contacting the acid addition salt so formed with a base, and isolating the free base form of the compound in the conventional manner. The free base forms of compounds prepared according to a process of the present invention differ from their respective acid addition salt forms somewhat in certain physical properties such as solubility, crystal structure, hygroscopicity, and the like, but otherwise free base forms of the compounds and their respective acid addition salt forms are equivalent for purposes of the present invention.

A pharmaceutically acceptable base addition salt of an acidic compound useful in the method of the present invention may be prepared by contacting the free acid form of the compound with a nontoxic metal cation such as an alkali or alkaline earth metal cation, or an amine, especially an organic amine. Examples of suitable metal cations include sodium cation (Na⁺), potassium cation (K⁺), magnesium cation (Mg²⁺), calcium cation (Ca²⁺), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of an acidic compound useful in the method of the present invention may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner. The free acid forms of the compounds useful in the method of the present invention differ from their respective salt forms somewhat in certain physical properties such as solubility, crystal structure, hygroscopicity, and the like, but otherwise the salts are equivalent to their respective free acid for purposes of the present invention.

Certain of the compounds useful in the method of the present invention can exist in unsolvated forms as well as solvated forms, including hydrated forms. In general, the solvated forms, including hydrated forms, are equivalent to unsolvated forms and are intended to be encompassed within the scope of the present invention.

Certain of the compounds useful in the method of the present invention possess one or more chiral centers, and each center may exist in the R or S configuration. A method of the present invention may utilize any diastereomeric, enantiomeric, or epimeric form of an alpha2delta ligand, or a pharmaceutically acceptable salt thereof, as well as mixtures thereof.

Additionally, certain compounds useful in the method of the present invention may exist as geometric isomers such as the entgegen (E) and zusammen (Z) isomers of alkenyl groups. A method of the present invention may utilize any cis, trans, syn, anti, entgegen (E), or zusammen (Z) isomer of an alpha2delta ligand, or a pharmaceutically acceptable salt thereof, as well as mixtures thereof.

Certain compounds useful in the method of the present invention can exist as two or more tautomeric forms. Tautomeric forms of the compounds may interchange, for example, via enolization/de-enolization and the like. A method of the present invention may utilize any tautomeric form of an alpha2delta ligand, or a pharmaceutically acceptable salt thereof, as well as mixtures thereof.

The following examples illustrate the invention pharmaceutical compositions containing a ADHD treating effective amount of an alpha2delta ligand, and a pharmaceutically acceptable carrier, diluent, or excipient. The examples are representative only, and are not to be construed as limiting the invention in any respect.

### FORMULATION EXAMPLE 1

Tablet Formulation:

| Ingredient | Amount (mg) |
|---|---|
| 3-(1-aminomethyl-cyclohexylmethyl)-4H- | 25 |
| [1,2,4]oxadiazol-5-one hydrochloride | |
| Lactose | 50 |
| Cornstarch (for mix) | 10 |
| Cornstarch (paste) | 10 |
| Magnesium stearate (1%) | 5 |
| Total | 100 |

3-(1-Aminomethyl-cyclohexylmethyl)-4H-[1,2,4] oxadiazol-5-one, hydrochloride, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of ADHD.

### FORMULATION EXAMPLE 2

Coated Tablets:

The tablets of Formulation Example 1 are coated in a customary manner with a coating of sucrose, potato starch, talc, tragacanth, and colorant.

### FORMULATION EXAMPLE 3

Injection Vials:

The pH of a solution of 500 g of gabapentin and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of gabapentin.

### FORMULATION EXAMPLE 4

Suppositories:

A mixture of 25 g of (1α,3α,5α)(3-aminomethyl-bicyclo[3.2.0]hept-3-yl)-acetic acid hydrochloride, 100 g of soya lecithin, and 1400 g of cocoa butter is fused, poured into molds, and allowed to cool. Each suppository contains 25 mg of (1α,3α,5α)(3-aminomethyl-bicyclo[3.2.0]hept-3-yl)-acetic acid hydrochloride.

### FORMULATION EXAMPLE 5

Solution:

A solution is prepared from 1 g of 3-(2-aminomethyl-4-methyl-pentyl)-4H-[1,2,4]-oxadiazol-5-one hydrochloride, 9.38 g of NaH₂PO₄.12H₂O, 28.48 g of Na₂HPO₄.12H₂O, and 0.1 g benzalkonium chloride in 940 mL of double-distilled water. The pH of the solution is adjusted to pH 6.8 using 2 M hydrochloric acid. The solution is diluted to 1.0 L with double-distilled water, and sterilized by irradiation. A 25 mL volume of the solution contains 25 mg of 3-(2-aminomethyl-4-methyl-pentyl)-4H-[1,2,4]-oxadiazol-5-one hydrochloride.

### FORMULATION EXAMPLE 6

Ointment:

500 mg of 3-(1-aminomethyl-cycloheptylmethyl)-4H-[1,2,4]oxadiazol-5-one hydrochloride is mixed with 99.5 g of petroleum jelly under aseptic conditions. A 5 g portion of the ointment contains 25 mg of 3-(1-aminomethyl-cycloheptylmethyl)-4H-[1,2,4]oxadiazol-5-one hydrochloride.

### FORMULATION EXAMPLE 7

Capsules:

2 kg of 3-(1-aminomethyl-cyclohexylmethyl)-4H-[1,2,4]oxadiazol-5-one hydrochloride are filled into hard gelatin capsules in a customary manner such that each capsule contains 25 mg of 3-(1-aminomethyl-cyclohexylmethyl)-4H-[1,2,4]oxadiazol-5-one hydrochloride.

### FORMULATION EXAMPLE 8

Ampoules:

A solution of 2.5 kg of gabapentin is dissolved in 60 L of double-distilled water. The solution is sterile filtered, and the filtrate is filled into ampoules. The ampoules are lyophilized under sterile conditions and aseptically sealed. Each ampoule contains 25 mg of gabapentin.

Having described the invention method, various embodiments of the invention are hereupon claimed.

What is claimed is:

1. A method of treating ADHD in a mammal suffering therefrom, comprising administering to a mammal in need of such treatment a therapeutically effective amount of an alpha2delta ligand or a pharmaceutically acceptable salt thereof.

2. A compound according to claim 2, wherein the alpha2delta ligand is gabapentin.

3. The method according to claim 1, wherein the alpha2delta ligand is a compound of Formula I 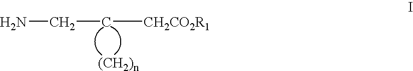
and pharmaceutically acceptable salts thereof, wherein R₁ is hydrogen or straight or branched lower alkyl, and n is an integer of from 4 to 6.

4. The method according to claim 1, wherein the alpha2delta ligand is a compound of Formula II 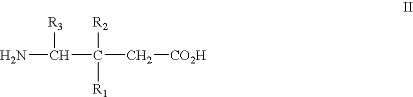
and pharmaceutically acceptable salts thereof, wherein:
R₁ is straight or branched unsubstituted alkyl of from 1 to 6 carbon atoms, unsubstituted phenyl, or unsubstituted cycloalkyl of from 3 to 6 carbon atoms;
R₂ is hydrogen or methyl; and
R₃ is hydrogen, methyl, or carboxyl.

5. The method according to claim 4, wherein the alpha2delta ligand is pregabalin.

6. The method according to claim 4, wherein the alpha2delta ligand is R-(3)-(aminomethyl)-5-methyl-hexanoic acid.

7. The method according to claim 4, wherein the alpha2delta ligand is 3-(1-aminoethyl)-5-methylheptanoic acid or 3-(1-aminoethyl)-5-methylhexanoic acid.

8. The method according to claim 1, wherein the alpha2delta ligand is a compound of the Formula 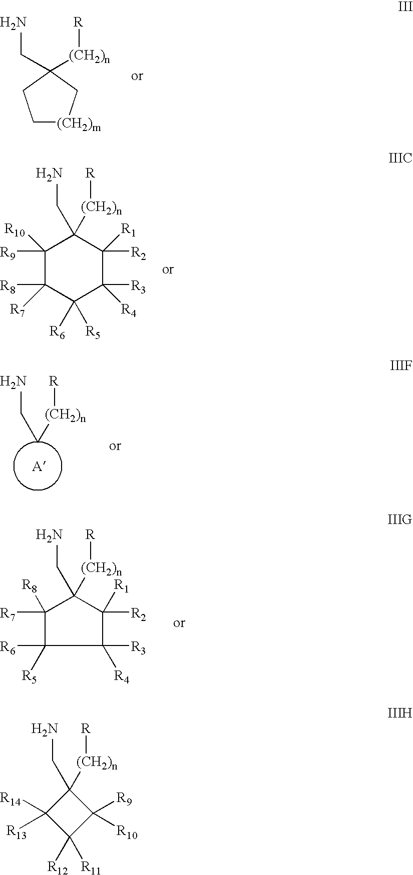
or a pharmaceutically acceptable salt thereof wherein:
n is an integer of from 0 to 2;
m is an integer of from 0 to 3;
R is sulfonamide,
amide,
phosphonic acid,
heterocycle,
sulfonic acid, or
hydroxamic acid;
R₁ to R₁₄ are each independently selected from hydrogen or straight or branched alkyl of from 1 to 6 carbons, unsubstituted or substituted benzyl or phenyl which substituents are selected from halogen, alkyl, alkoxy, hydroxy, carboxy, carboalkoxy, trifluoromethyl, and nitro;
A′ is a bridged ring selected from 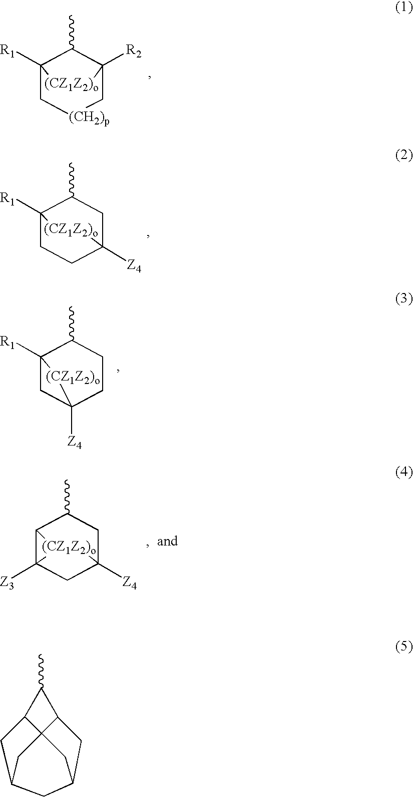
wherein 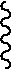
is the point of attachment;
Z₁ to Z₄ are each independently selected from hydrogen and methyl;
o is an integer of from 1 to 4; and
p is an integer of from 0 to 2 with the proviso that in formula 1 R is not —SO₃H when m is 2 and n is 1.

9. The method according to claim 8, wherein the alpha2delta ligand is a compound of Formula III 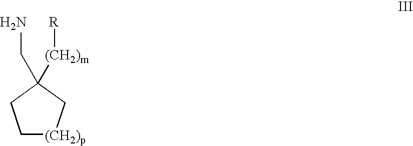
and pharmaceutically acceptable salts thereof, wherein:
m is an integer of from 0 to 2;
p is an integer of from 0 to 3; and
R is sulfonamide,
amide,
phosphonic acid,
heterocycle,
sulfonic acid, or
hydroxamic acid.

10. The method according to claim 8, wherein the alpha2delta ligand is a compound of Formula III 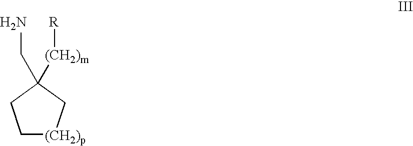
and pharmaceutically acceptable salts thereof, wherein:
m is an integer of from 0 to 2;
p is an integer of 2; and

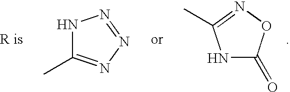

11. The method according to claim 8, wherein the alpha2delta ligand is 3-(1-aminomethyl-cyclohexylmethyl)-4H-[1,2,4]oxadiazol-5-one, or a pharmaceutically acceptable salt thereof.

12. The method according to claim 8, wherein the alpha2delta ligand is 3-(1-aminomethyl-cyclohexylmethyl)-4H-[1,2,4]oxadiazol-5-one hydrochloride.

13. The method according to claim 8, wherein the alpha2delta ligand is 3-(1-aminomethyl-cycloheptylmethyl)-4H-[1,2,4]oxadiazol-5-one, or a pharmaceutically acceptable salt thereof.

14. The method according to claim 8, wherein the alpha2delta ligand is 3-(1-aminomethyl-cycloheptylmethyl)-4H-[1,2,4]oxadiazol-5-one hydrochloride.

15. The method according to claim 8, wherein the alpha2delta ligand is C-[1-(1H-tetrazol-5-ylmethyl)-cycloheptyl]-methylamine, or a pharmaceutically acceptable salt thereof.

16. The method according to claim 8, wherein the alpha2delta ligand is C-[1-(1H-tetrazol-5-ylmethyl)-cycloheptyl]-methylamine.

17. The method according to claim 8, wherein the alpha2delta ligand is a compound of the Formula III, IIIC, IIIF, IIIG, or IIIH, wherein R is a sulfonamide selected from —NHSO₂R¹⁵ or —SO₂NHR¹⁵ wherein R¹⁵ is straight or branched alkyl or trifluoromethyl.

18. The method according to claim 8, wherein the alpha2delta ligand is N-[2-(1-aminomethyl-cyclohexyl)-ethyl]-methanesulfonamide.

19. The method according to claim 8, wherein the alpha2delta ligand is a compound of the Formula III, IIIC, IIIF, IIIG, or IIIH wherein R is a phosphonic acid, —PO₃H₂.

20. The method according to claim 8, wherein the alpha2delta ligand is selected from (1-aminomethyl-cyclohexylmethyl)-phosphonic acid and (2-aminomethyl-4-methyl-pentyl)-phosphonic acid.

21. The method according to claim 8, wherein the alpha2delta ligand is a compound of the Formula III, IIIC, IIIF, IIIG, or IIIH wherein R is a heterocycle selected from

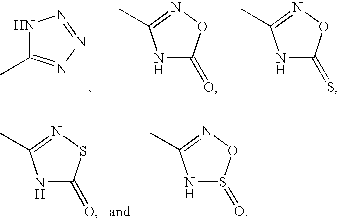

22. The method according to claim 8, wherein the alpha2delta ligand is selected from C-[1-(1H-tetrazol-5-ylmethyl)cyclohexyl]-methylamine and 4-methyl-2-(1H-tetrazol-5-ylmethyl)-pentylamine.

23. The method according to claim 8, wherein the alpha2delta ligand is selected from:
(1-Aminomethyl-cyclohexylmethyl)-phosphonic acid;
(1R-trans)(1-Aminomethyl-3-methyl-cyclohexylmethyl)-phosphonic acid;
(trans)(1-Aminomethyl-3,4-dimethyl-cyclopentylmethyl)-phosphonic acid;
(1R-trans)(1-Aminomethyl-3-methyl-cyclopentylmethyl)-phosphonic acid;
(1S-cis)(1-Aminomethyl-3-methyl-cyclopentylmethyl)-phosphonic acid;
(1S-trans)(1-Aminomethyl-3-methyl-cyclopentylmethyl)-phosphonic acid;
(1R-cis)(1-Aminomethyl-3-methyl-cyclopentylmethyl)-phosphonic acid;
(1α,3α,4α)(1-Aminomethyl-3,4-dimethyl-cyclopentylmethyl)-phosphonic acid;
(1α,3β,4β)(1-Aminomethyl-3,4-dimethyl-cyclopentylmethyl)-phosphonic acid;
(R)(1-Aminomethyl-3,3-dimethyl-cyclopentylmethyl)-phosphonic acid;
(S)(1-Aminomethyl-3,3-dimethyl-cyclopentylmethyl)-phosphonic acid;
(1-Aminomethyl-3,3-dimethyl-cyclobutylmethyl)-phosphonic acid;
2-(1-Aminomethyl-cyclohexyl)-N-hydroxy-acetamide;
(1S-trans)2-(1-Aminomethyl-3-methyl-cyclohexyl)-N-hydroxy-acetamide;
(trans)2-(1-Aminomethyl-3,4-dimethyl-cyclopentyl)-N-hydroxy-acetamide;
(1S-cis)2-(1-Aminomethyl-3-methyl-cyclopentyl)-N-hydroxy-acetamide;
(1R-trans)2-(1-Aminomethyl-3-methyl-cyclopentyl)-N-hydroxy-acetamide;
(1R-cis)2-(1-Aminomethyl-3-methyl-cyclopentyl)-N-hydroxy-acetamide;
(1S-trans)2-(1-Aminomethyl-3-methyl-cyclopentyl)-N-hydroxy-acetamide;
(1α,3α,4α)2-(1-Aminomethyl-3,4-dimethyl-cyclopentyl)-N-hydroxy-acetamide;
(1α,3β,4β)2-(1-Aminomethyl-3,4-dimethyl-cyclopentyl)-N-hydroxy-acetamide;
(S)2-(1-Aminomethyl-3,3-dimethyl-cyclopentyl)-N-hydroxy-acetamide;
(R)2-(1-Aminomethyl-3,3-dimethyl-cyclopentyl)-N-hydroxy-acetamide;
2-(1-Aminomethyl-3,3-dimethyl-cyclobutyl)-N-hydroxy-acetamide;
N-[2-(1-Aminomethyl-cyclohexyl)-ethyl]-methanesulfonamide;
(1S-cis)N-[2-(1-Aminomethyl-3-methyl-cyclohexyl)-ethyl]-methanesulfonamide;
(trans)N-[2-(1-Aminomethyl-3,4-dimethyl-cyclopentyl)-ethyl]-methanesulfonamide;
(1S-cis)N-[2-(1-Aminomethyl-3-methyl-cyclopentyl)-ethyl]-methanesulfonamide;
(1R-trans)N-[2-(1-Aminomethyl-3-methyl-cyclopentyl)-ethyl]-methanesulfonamide;
(1R-cis)N-[2-(1-Aminomethyl-3-methyl-cyclopentyl)-ethyl]-methanesulfonamide;
(1S-cis)N-[2-(1-Aminomethyl-3-methyl-cyclopentyl)-ethyl]-methanesulfonamide;
(1α,3α,4α)N-[2-(1-Aminomethyl-3,4-dimethyl-cyclopentyl)-ethyl]-methanesulfonamide;
(1α,3β,4β)N-[2-(1-Aminomethyl-3,4-dimethyl-cyclopentyl)-ethyl]-methanesulfonamide;
(S)N-[2-(1-Aminomethyl-3,3-dimethyl-cyclopentyl)-ethyl]-methanesulfonamide;
(R)N-[2-(1-Aminomethyl-3,3-dimethyl-cyclopentyl)-ethyl]-methanesulfonamide;
N-[2-(1-Aminomethyl-3,3-dimethyl-cyclobutyl)-ethyl]-methanesulfonamide;
(1S-cis)3-(1-Aminomethyl-3-methyl-cyclohexylmethyl)-4H-[1,2,4]oxadiazol-5-one;
(trans)3-(1-Aminomethyl-3,4-dimethyl-cyclopentylmethyl)-4H-[1,2,4]oxadiazol-5-one;
(1S-cis)3-(1-Aminomethyl-3-methyl-cyclopentylmethyl)-4H-[1,2,4]oxadiazol-5-one;
(1R-trans)3-(1-Aminomethyl-3-methyl-cyclopentylmethyl)-4H-[1,2,4]oxadiazol-5-one;
(1R-cis)3-(1-Aminomethyl-3-methyl-cyclopentylmethyl)-4H-[1,2,4]oxadiazol-5-one;
(1S-trans)3-(1-Aminomethyl-3-methyl-cyclopentylmethyl)-4H-[1,2,4]oxadiazol-5-one;
(1α,3α,4α)3-(1-Aminomethyl-3,4-dimethyl-cyclopentylmethyl)-4H-[1,2,4]oxadiazol-5-one;
(1α,3β,4β)3-(1-Aminomethyl-3,4-dimethyl-cyclopentylmethyl)-4H-[1,2,4]oxadiazol-5-one;
(S)3-(1-Aminomethyl-3,3-dimethyl-cyclopentylmethyl)-4H-[1,2,4]oxadiazol-5-one;
(R)3-(1-Aminomethyl-3,3-dimethyl-cyclopentyl methyl)-4H-[1,2,4]oxadiazol-5-one;
3-(1-Aminomethyl-3,3-dimethyl-cyclobutylmethyl)-4H-[1,2,4]oxadiazol-5-one;
3-(1-Aminomethyl-cyclohexylmethyl)-4H-[1,2,4]oxadiazole-5-thione;
(1S-cis)3-(1-Aminomethyl-3-methyl-cyclohexylmethyl)-4H-[1,2,4]oxadiazole-5-thione;
(trans)3-(1-Aminomethyl-3,4-dimethyl-cyclopentylmethyl)-4H-[1,2,4]oxadiazole-5-thione;
(1S-cis)3-(1-Aminomethyl-3-methyl-cyclopentylmethyl)-4H-[1,2,4]oxadiazole-5-thione;
(1R-trans)3-(1-Aminomethyl-3-methyl-cyclopentylmethyl)-4H-[1,2,4]oxadiazole-5-thione;
(1R-cis)3-(1-Aminomethyl-3-methyl-cyclopentylmethyl)-4H-[1,2,4]oxadiazole-5-thione;
(1S-trans)3-(1-Aminomethyl-3-methyl-cyclopentylmethyl)-4H-[1,2,4]oxadiazole-5-thione;
(1α,3α,4α)3-(1-Aminomethyl-3,4-dimethyl-cyclopentylmethyl)-4H-[1,2,4]oxadiazole-5-thione;
(1α,3β,4β)3-(1-Aminomethyl-3,4-dimethyl-cyclopentylmethyl)-4H-[1,2,4]oxadiazole-5-thione;
(S)3-(1-Aminomethyl-3,3-dimethyl-cyclopentylmethyl)-4H-[1,2,4]oxadiazole-5-thione;
(R)3-(1-Aminomethyl-3,3-dimethyl-cyclopentylmethyl)-4H-[1,2,4]oxadiazole-5-thione;
3-(1-Aminomethyl-3,3-dimethyl-cyclobutylmethyl)-4H-[1,2,4]oxadiazole-5-thione;
C-[1-(1H-Tetrazol-5-ylmethyl)-cyclohexyl]-methylamine;
(1S-cis)C-[3-Methyl-1-(1H-tetrazol-5-ylmethyl)-cyclohexyl]-methylamine;
(trans)C-[3,4-Dimethyl-1-(1H-tetrazol-5-ylmethyl)-cyclopentyl]-methylamine;
(1S-cis)C-[3-Methyl-1-(1H-tetrazol-5-ylmethyl)-cyclopentyl]-methylamine;
(1R-trans)C-[3-Methyl-1-(1H-tetrazol-5-ylmethyl)-cyclopentyl]-methylamine;
(1R-cis)C-[3-Methyl-1-(1H-tetrazol-5-ylmethyl)-cyclopentyl]-methylamine;
(1S-trans)C-[3-Methyl-1-(1H-tetrazol-5-ylmethyl)-cyclopentyl]-methylamine;
(1α,3α,4α)-[3,4-Dimethyl-1-(1H-tetrazol-5-ylmethyl)-cyclopentyl]-methylamine;
(1α,3β,4β)-[3,4-Dimethyl-1-(1H-tetrazol-5-ylmethyl)-cyclopentyl]-methylamine;
(S)C-[3,3-Dimethyl-1-(1H-tetrazol-5-ylmethyl)-cyclopentyl]-methylamine;
(R)C-[3,3-Dimethyl-1-(1H-tetrazol-5-ylmethyl)-cyclopentyl]-methylamine;
C-[3,3-Dimethyl-1-(1H-tetrazol-5-ylmethyl)-cyclobutyl]-methylamine;
N-[2-(1-Aminomethyl-cyclohexyl)-ethyl]-C,C,C-trifluoro-methanesulfonamide;
(1S-cis)N-[2-(1-Aminomethyl-3-methyl-cyclohexyl)-ethyl]-C,C,C-trifluoro-methanesulfonamide;
(trans)N-[2-(1-Aminomethyl-3,4-dimethyl-cyclopentyl)-ethyl]-C,C,C-trifluoro-methanesulfonamide;
(1R-cis)N-[2-(1-Aminomethyl-3-methyl-cyclopentyl)-ethyl]-C,C,C-trifluoro-methanesulfonamide;
(1S-trans)N-[2-(1-Aminomethyl-3-methyl-cyclopentyl)-ethyl]-C,C,C-trifluoro-methanesulfonamide;
(1S-cis)N-[2-(1-Aminomethyl-3-methyl-cyclopentyl)-ethyl]-C,C,C-trifluoro-methanesulfonamide;
(1R-trans)N-[2-(1-Aminomethyl-3-methyl-cyclopentyl)-ethyl]-C,C,C-trifluoro-methanesulfonamide;
(1α,3α,4α)N-[2-(1-Aminomethyl-3,4-dimethyl-cyclopentyl)-ethyl]-C,C,C-trifluoro-methanesulfonamide;
(1α,3β,4β)N-[2-(1-Aminomethyl-3,4-dimethyl-cyclopentyl)-ethyl]-C,C,C-trifluoro-methanesulfonamide;
(S)N-[2-(1-Aminomethyl-3,3-dimethyl-cyclopentyl)-ethyl]-C,C,C-trifluoro-methanesulfonamide;
(R)N-[2-(1-Aminomethyl-3,3-dimethyl-cyclopentyl)-ethyl]-C,C,C-trifluoro-methanesulfonamide;
N-[2-(1-Aminomethyl-3,3-dimethyl-cyclobutyl)-ethyl]-C,C,C-trifluoro-methanesulfonamide;
3-(1-Aminomethyl-cyclohexylmethyl)-4H-[1,2,4]thiadiazol-5-one;
(1S-cis)3-(1-Aminomethyl-3-methyl-cyclohexylmethyl)-4H-[1,2,4]thiadiazol-5-one;
(trans)3-(1-Aminomethyl-3,4-dimethyl-cyclopentylmethyl)-4H-[1,2,4]thiadiazol-5-one;
(1R-cis)3-(1-Aminomethyl-3-methyl-cyclopentylmethyl)-4H-[1,2,4]thiadiazol-5-one;
(1S-trans)3-(1-Aminomethyl-3-methyl-cyclopentylmethyl)-4H-[1,2,4]thiadiazol-5-one;
(1S-cis)3-(1-Aminomethyl-3-methyl-cyclopentylmethyl)-4H-[1,2,4]thiadiazol-5-one;
(1R-trans)3-(1-Aminomethyl-3-methyl-cyclopentylmethyl)-4H-[1,2,4]thiadiazol-5-one;
(1α,3α,4α)3-(1-Aminomethyl-3,4-dimethyl-cyclopentylmethyl)-4H-[1,2,4]thiadiazol-5-one;
(1α,3β,4β)3-(1-Aminomethyl-3,4-dimethyl-cyclopentylmethyl)-4H-[1,2,4]thiadiazol-5-one;
(S)3-(1-Aminomethyl-3,3-dimethyl-cyclopentylmethyl)-4H-[1,2,4]thiadiazol-5-one;
(R)3-(1-Aminomethyl-3,3-dimethyl-cyclopentylmethyl)-4H-[1,2,4]thiadiazol-5-one;
3-(1-Aminomethyl-3,3-dimethyl-cyclobutylmethyl)-4H-[1,2,4]thiadiazol-5-one;
C-[1-(2-Oxo-2,3-dihydro-2λ⁴-[1,2,3,5]oxathiadiazol-4-ylmethyl)-cyclohexyl]-methylamine;
(1S-cis)C-[3-Methyl-1-(2-oxo-2,3-dihydro-2λ⁴-[1,2,3,5]oxathiadiazol-4-ylmethyl)-cyclohexyl]-methylamine;
(trans)C-[3,4-Dimethyl-1-(2-oxo-2,3-dihydro-2λ⁴-[1,2,3,5]oxathiadiazol-4-ylmethyl)-cyclopentyl]-methylamine;
(1S-cis)C-[3-Methyl-1-(2-oxo-2,3-dihydro-2λ⁴-[1,2,3,5]oxathiadiazol-4-ylmethyl)-cyclopentyl]-methyl amine;
(1R-trans)C-[3-Methyl-1-(2-oxo-2,3-dihydro-2λ⁴-[1,2,3,5]oxathiadiazol-4-ylmethyl)-cyclopentyl]-methylamine;
(1R-cis)C-[3-Methyl-1-(2-oxo-2,3-dihydro-2λ⁴-[1,2,3,5]oxathiadiazol-4-ylmethyl)-cyclopentyl]-methylamine;
(1S-trans)C-[3-Methyl-1-(2-oxo-2,3-dihydro-2λ⁴-[1,2,3,5]oxathiadiazol-4-ylmethyl)-cyclopentyl]-methylamine;
(1α,3α,4α)C-[3,4-Dimethyl-1-(2-oxo-2,3-dihydro-2λ⁴-[1,2,3,5]oxathiadiazol-4-ylmethyl)-cyclopentyl]-methylamine;
(1λ,3β,4β)C-[3,4-Dimethyl-1-(2-oxo-2,3-dihydro-2λ⁴-[1,2,3,5]oxathiadiazol-4-ylmethyl)-cyclopentyl]-methylamine;
(S)C-[3,3-Dimethyl-1-(2-oxo-2,3-dihydro-2λ⁴-[1,2,3,5]oxathiadiazol-4-ylmethyl)-cyclopentyl]-methylamine;
(R)C-[3,3-Dimethyl-1-(2-oxo-2,3-dihydro-2λ⁴-[1,2,3,5]oxathiadiazol-4-ylmethyl)-cyclopentyl]-methylamine;
C-[3,3-Dimethyl-1-(2-oxo-2,3-dihydro-2λ⁴-[1,2,3,5]oxathiadiazol-4-ylmethyl)-cyclobutyl]-methylamine;
(1-Aminomethyl-cyclohexyl)-methanesulfonamide;
(1R-trans)(1-Aminomethyl-3-methyl-cyclohexyl)-methanesulfonamide;
(trans)(1-Aminomethyl-3,4-dimethyl-cyclopentyl)-methanesulfonamide;
(1S-trans)(1-Aminomethyl-3-methyl-cyclopentyl)-methanesulfonamide;
(1R-cis)(1-Aminomethyl-3-methyl-cyclopentyl)-methanesulfonamide;
(1R-trans)(1-Aminomethyl-3-methyl-cyclopentyl)-methanesulfonamide;
(1S-cis)(1-Aminomethyl-3-methyl-cyclopentyl)-methanesulfonamide;
(1α,3β,4β)(1-Aminomethyl-3,4-dimethyl-cyclopentyl)-methanesulfonamide;
(1α,3α,4α)(1-Aminomethyl-3,4-dimethyl-cyclopentyl)-methanesulfonamide;
(R)(1-Aminomethyl-3,3-dimethyl-cyclopentyl)-methanesulfonamide;
(S)(1-Aminomethyl-3,3-dimethyl-cyclopentyl)-methanesulfonamide;
(1-Aminomethyl-3,3-dimethyl-cyclobutyl)-methanesulfonamide;
(1-Aminomethyl-cyclohexyl)-methanesulfonic acid;
(1R-trans)(1-Aminomethyl-3-methyl-cyclohexyl)-methanesulfonic acid;
(trans)(1-Aminomethyl-3,4-dimethyl-cyclopentyl)-methanesulfonic acid;
(1S-trans)(1-Aminomethyl-3-methyl-cyclopentyl)-methanesulfonic acid;
(1S-cis)(1-Aminomethyl-3-methyl-cyclopentyl)-methanesulfonic acid;
(1R-trans)(1-Aminomethyl-3-methyl-cyclopentyl)-methanesulfonic acid;
(1R-cis)(1-Aminomethyl-3-methyl-cyclopentyl)-methanesulfonic acid;
(1α,3β,4β)(1-Aminomethyl-3,4-dimethyl-cyclopentyl)-methanesulfonic acid;
(1α,3α,4α)(1-Aminomethyl-3,4-dimethyl-cyclopentyl)-methanesulfonic acid;
(R)(1-Aminomethyl-3,3-dimethyl-cyclopentyl)-methanesulfonic acid;
(S)(1-Aminomethyl-3,3-dimethyl-cyclopentyl)-methanesulfonic acid;
(1-Aminomethyl-3,3-dimethyl-cyclobutyl)-methanesulfonic acid;
(1-Aminomethyl-cyclopentylmethyl)-phosphonic acid;
2-(1-Aminomethyl-cyclopentyl)-N-hydroxy-acetamide;
N-[2-(1-Aminomethyl-cyclopentyl)-ethyl]-methanesulfonamide;
3-(1-Aminomethyl-cyclopentylmethyl)-4H-[1,2,4]oxadiazol-5-one;
3-(1-Aminomethyl-cyclopentylmethyl)-4H-[1,2,4]oxadiazole-5-thione;
C-[1-(1H-Tetrazol-5-ylmethyl)-cyclopentyl]-methylamine;
N-[2-(1-Aminomethyl-cyclopentyl)-ethyl]-C,C,C-trifluoro-methanesulfonamide;
3-(1-Aminomethyl-cyclopentylmethyl)-4H-[1,2,4]thiadiazol-5-one;
C-[1-(2-Oxo-2,3-dihydro-2λ⁴-[1,2,3,5]oxathiadiazol-4-ylmethyl)-cyclopentyl]-methylamine;
(1-Aminomethyl-cyclopentyl)-methanesulfonamide;
(1-Aminomethyl-cyclopentyl)-methanesulfonic acid;
(9-Aminomethyl-bicyclo[3.3.1]non-9-ylmethyl)-phosphonic acid;
2-(9-Aminomethyl-bicyclo[3.3.1]non-9-yl)-N-hydroxy-acetamide;
N-[2-(9-Aminomethyl-bicyclo[3.3.1]non-9-yl)-ethyl]-methanesulfonamide;
3-(9-Aminomethyl-bicyclo[3.3.1]non-9-ylmethyl)-4H-[1,2,4]oxadiazol-5-one;
3-(9-Aminomethyl-bicyclo[3.3.1]non-9-ylmethyl)-4H-[1,2,4]oxadiazole-5-thione;
C-[9-(1H-Tetrazol-5-ylmethyl)-bicyclo[3.3.1]non-9-yl]-methylamine;
N-[2-(9-Aminomethyl-bicyclo[3.3.1]non-9-yl)-ethyl]-C,C,C-trifluoro-methanesulfonamide;
3-(9-Aminomethyl-bicyclo[3.3.1]non-9-ylmethyl)-4H-[1,2,4]thiadiazol-5-one;
C-[9-(2-Oxo-2,3-dihydro-2λ⁴-[1,2,3,5]oxathiadiazol-4-ylmethyl)-bicyclo[3.3.1]non-9-yl]-methylamine;
(9-Aminomethyl-bicyclo[3.3.1]non-9-yl)-methanesulfonamide;
(9-Aminomethyl-bicyclo[3.3.1]non-9-yl)-methanesulfonic acid;
(2-Aminomethyl-adamantan-2-ylmethyl)-phosphonic acid;
2-(2-Aminomethyl-adamantan-2-yl)-N-hydroxy-acetamide;
N-[2-(2-Aminomethyl-adamantan-2-yl)-ethyl]-methanesulfonamide;
3-(2-Aminomethyl-adamantan-2-ylmethyl)-4H-[1,2,4]oxadiazol-5-one;
3-(2-Aminomethyl-adamantan-2-ylmethyl)-4H-[1,2,4]oxadiazole-5-thione;
C-[2-(1H-Tetrazol-5-ylmethyl)-adamantan-2-yl]-methylamine;
N-[2-(2-Aminomethyl-adamantan-2-yl)-ethyl]-C,C,C-trifluoro-methanesulfonamide;
3-(2-Aminomethyl-adamantan-2-ylmethyl)-4H-[1,2,4]thiadiazol-5-one;
C-[2-(2-Oxo-2,3-dihydro-2λ⁴-[1,2,3,5]oxathiadiazol-4-ylmethyl)-adamantan-2-yl]-methylamine;
(2-Aminomethyl-adamantan-2-yl)-methanesulfonamide;
(2-Aminomethyl-adamantan-2-yl)-methanesulfonic acid
(1-Aminomethyl-cycloheptylmethyl)-phosphonic acid;
2-(1-Aminomethyl-cycloheptyl)-N-hydroxy-acetamide;
N-[2-(1-Aminomethyl-cycloheptyl)-ethyl]-methanesulfonamide;
3-(1-Aminomethyl-cycloheptylmethyl)-4H-[1,2,4]oxadiazole-5-thione;
N-[2-(1-Aminomethyl-cycloheptyl)-ethyl]-C,C,C-trifluoro-methanesulfonamide;
C-[1-(2-Oxo-2,3-dihydro-2,4-[1,2,3,5]oxathiadiazol-4-ylmethyl)-cycloheptyl]-methylamine;
(1-Aminomethyl-cycloheptyl)-methanesulfonamide; and
(1-Aminomethyl-cycloheptyl)-methanesulfonic acid.

24. The method according to claim 1, wherein the alpha2delta ligand is a compound of Formula IV 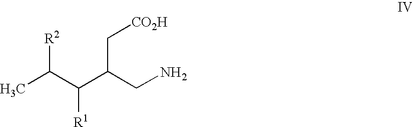
or a pharmaceutically acceptable salt thereof wherein:
R₁ is hydrogen, straight or branched alkyl of from 1 to 6 carbon atoms or phenyl;
R² is straight or branched alkyl of from 1 to 8 carbon atoms, straight or branched alkenyl of from 2 to 8 carbon atoms, cycloalkyl of from 3 to 7 carbon atoms, alkoxy of from 1 to 6 carbon atoms,
-alkylcycloalkyl,
-alkylalkoxy,
-alkyl OH,
-alkylphenyl,
-alkylphenoxy,
-phenyl or substituted phenyl; and
R¹ is straight or branched alkyl of from 1 to 6 carbon atoms or phenyl when R² is methyl.

25. The method according to claim 24, wherein the alpha2delta ligand is a compound of Formula IV wherein R¹ is hydrogen, and R² is alkyl.

26. The method according to claim 24, wherein the Alpha2delta ligand is a compound of Formula IV wherein R¹ is methyl, and R² is alkyl.

27. The method according to claim 24, wherein the Alpha2delta ligand is a compound of Formula IV wherein R¹ is methyl, and R² is methyl or ethyl.

28. The method according to claim 24, wherein the alpha2delta ligand is selected from:
3-Aminomethyl-5-methylheptanoic acid;
3-Aminomethyl-5-methyl-octanoic acid;
3-Aminomethyl-5-methyl-nonanoic acid;
3-Aminomethyl-5-methyl-decanoic acid;
3-Aminomethyl-5-methyl-undecanoic acid;
3-Aminomethyl-5-methyl-dodecanoic acid;
3-Aminomethyl-5-methyl-tridecanoic acid;
3-Aminomethyl-5-cyclopropyl-hexanoic acid;
3-Aminomethyl-5-cyclobutyl-hexanoic acid;
3-Aminomethyl-5-cyclopentyl-hexanoic acid;
3-Aminomethyl-5-cyclohexyl-hexanoic acid;
3-Aminomethyl-5-trifluoromethyl-hexanoic acid;
3-Aminomethyl-5-phenyl-hexanoic acid;
3-Aminomethyl-5-(2-chlorophenyl)-hexanoic acid;
3-Aminomethyl-5-(3-chlorophenyl)-hexanoic acid;
3-Aminomethyl-5-(4-chlorophenyl)-hexanoic acid;
3-Aminomethyl-5-(2-methoxyphenyl)-hexanoic acid;
3-Aminomethyl-5-(3-methoxyphenyl)-hexanoic acid;
3-Aminomethyl-5-(4-methoxyphenyl)-hexanoic acid; and
3-Aminomethyl-5-(phenylmethyl)-hexanoic acid.

29. The method according to claim 24, wherein the alpha2delta ligand is selected from:
(3R,4S)3-Aminomethyl-4,5-dimethyl-hexanoic acid;
3-Aminomethyl-4,5-dimethyl-hexanoic acid;
(3R,4S)3-Aminomethyl-4,5-dimethyl-hexanoic acid MP;
(3S,4S)3-Aminomethyl-4,5-dimethyl-hexanoic acid;
(3R,4R)3-Aminomethyl-4,5-dimethyl-hexanoic acid MP;
3-Aminomethyl-4-isopropyl-hexanoic acid;
3-Aminomethyl-4-isopropyl-heptanoic acid;
3-Aminomethyl-4-isopropyl-octanoic acid;
3-Aminomethyl-4-isopropyl-nonanoic acid;
3-Aminomethyl-4-isopropyl-decanoic acid; and
3-Aminomethyl-4-phenyl-5-methyl-hexanoic acid.

30. The method according to claim 24, wherein the alpha2delta ligand is (3S,5R)-3-Aminomethyl-5-methyl-heptanoic acid.

31. The method according to claim 24, wherein the alpha2delta ligand is (3S,5R)-3-Aminomethyl-5-methyl-octanoic acid.

32. The method according to claim 24, wherein the alpha2delta ligand is (3S,5R)-3-Aminomethyl-5-methyl-nonanoic acid.

33. The method according to claim 24, wherein the alpha2delta ligand is (3S,5R)-3-Aminomethyl-5-methyl-decanoic acid.

34. The method according to claim 24, wherein the alpha2delta ligand is (3S,5R)-3-Aminomethyl-5-methyl-undecanoic acid.

35. The method according to claim 24, wherein the alpha2delta ligand is (3S,5R)-3-Aminomethyl-5-methyl-dodecanoic acid.

36. The method according to claim 24, wherein the alpha2delta ligand is selected from:
(3S,5R)-3-Aminomethyl-5,9-dimethyl-decanoic acid;
(3S,5R)-3-Aminomethyl-5-methyl-heptanoic acid;
(3S,5R)-3-Aminomethyl-5,7-dimethyl-octanoic acid;
(3S,5R)-3-Aminomethyl-5,10-dimethyl-undecanoic acid;
(3S,5R)-3-Aminomethyl-5,8-dimethyl-nonanoic acid;
(3S,5R)-3-Aminomethyl-6-cyclopropyl-5-methyl-hexanoic acid;
(3S,5R)-3-Aminomethyl-6-cyclobutyl-5-methyl-hexanoic acid;
(3S,5R)-3-Aminomethyl-6-cyclopentyl-5-methyl-hexanoic acid;
(3S,5R)-3-Aminomethyl-6-cyclohexyl-5-methyl-hexanoic acid;
(3S,5R)-3-Aminomethyl-7-cyclopropyl-5-methyl-heptanoic acid;
(3S,5R)-3-Aminomethyl-7-cyclobutyl-5-methyl-heptanoic acid;
(3S,5R)-3-Aminomethyl-7-cyclopentyl-5-methyl-heptanoic acid;
(3S,5R)-3-Aminomethyl-7-cyclohexyl-5-methyl-heptanoic acid;
(3S,5R)-3-Aminomethyl-8-cyclopropyl-5-methyl-octanoic acid;
(3S,5R)-3-Aminomethyl-8-cyclobutyl-5-methyl-octanoic acid;
(3S,5R)-3-Aminomethyl-8-cyclopentyl-5-methyl-octanoic acid;
(3S,5R)-3-Aminomethyl-8-cyclohexyl-5-methyl-octanoic acid;
(3S,5S)-3-Aminomethyl-6-fluoro-5-methyl-hexanoic acid;
(3S,5S)-3-Aminomethyl-7-fluoro-5-methyl-heptanoic acid;
(3S,5R)-3-Aminomethyl-8-fluoro-5-methyl-octanoic acid;
(3S,5R)-3-Aminomethyl-9-fluoro-5-methyl-nonanoic acid;
(3S,5S)-3-Aminomethyl-7,7,7-trifluoro-5-methyl-heptanoic acid; and
(3S,5R)-3-Aminomethyl-8,8,8-trifluoro-5-methyl-octanoic acid.

37. The method according to claim 24, wherein the alpha2delta ligand is selected from:
(3S,5S)-3-Aminomethyl-5-methoxy-hexanoic acid;
(3S,5R)-3-Aminomethyl-8-hydroxy-5-methyl-octanoic acid;
(3S,5S)-3-Aminomethyl-5-ethoxy-hexanoic acid;
(3S,5S)-3-Aminomethyl-5-propoxy-hexanoic acid;
(3S,5S)-3-Aminomethyl-5-isopropoxy-hexanoic acid;
(3S,5S)-3-Aminomethyl-5-tert-butoxy-hexanoic acid;
(3S,5S)-3-Aminomethyl-5-fluoromethoxy-hexanoic acid;
(3S,5S)-3-Aminomethyl-5-(2-fluoro-ethoxy)-hexanoic acid;
(3S,5S)-3-Aminomethyl-5-(3,3,3-trifluoro-propoxy)-hexanoic acid;
(3S,5S)-3-Aminomethyl-5-phenoxy-hexanoic acid;
(3S,5S)-3-Aminomethyl-5-(4-chloro-phenoxy)-hexanoic acid;
(3S,5S)-3-Aminomethyl-5-(3-chloro-phenoxy)-hexanoic acid;
(3S,5S)-3-Aminomethyl-5-(2-chloro-phenoxy)-hexanoic acid;
(3S,5S)-3-Aminomethyl-5-(4-fluoro-phenoxy)-hexanoic acid;
(3S,5S)-3-Aminomethyl-5-(3-fluoro-phenoxy)-hexanoic acid;
(3S,5S)-3-Aminomethyl-5-(2-fluoro-phenoxy)-hexanoic acid;
(3S,5S)-3-Aminomethyl-5-(4-methoxy-phenoxy)-hexanoic acid;
(3S,5S)-3-Aminomethyl-5-(3-methoxy-phenoxy)-hexanoic acid;
(3S,5S)-3-Aminomethyl-5-(2-methoxy-phenoxy)-hexanoic acid;
(3S,5S)-3-Aminomethyl-5-(4-nitro-phenoxy)-hexanoic acid;
(3S,5S)-3-Aminomethyl-5-(3-nitro-phenoxy)-hexanoic acid;
(3S,5S)-3-Aminomethyl-5-(2-nitro-phenoxy)-hexanoic acid;
(3S,5S)-3-Aminomethyl-6-hydroxy-5-methyl-hexanoic acid;
(3S,5S)-3-Aminomethyl-6-methoxy-5-methyl-hexanoic acid;
(3S,5S)-3-Aminomethyl-6-ethoxy-5-methyl-hexanoic acid;
(3S,5S)-3-Aminomethyl-5-methyl-6-propoxy-hexanoic acid;
(3S,5S)-3-Aminomethyl-6-isopropoxy-5-methyl-hexanoic acid;
(3S,5S)-3-Aminomethyl-6-tert-butoxy-5-methyl-hexanoic acid;
(3S,5S)-3-Aminomethyl-6-fluoromethoxy-5-methyl-hexanoic acid;
(3S,5S)-3-Aminomethyl-6-(2-fluoro-ethoxy)-5-methyl-hexanoic acid;
(3S,5S)-3-Aminomethyl-5-methyl-6-(3,3,3-trifluoro-propoxy)-hexanoic acid;
(3S,5S)-3-Aminomethyl-5-methyl-6-phenoxy-hexanoic acid;
(3S,5S)-3-Aminomethyl-6-(4-chloro-phenoxy)-5-methyl-hexanoic acid;
(3S,5S)-3-Aminomethyl-6-(3-chloro-phenoxy)-5-methyl-hexanoic acid;
(3S,5S)-3-Aminomethyl-6-(2-chloro-phenoxy)-5-methyl-hexanoic acid;
(3S,5S)-3-Aminomethyl-6-(4-fluoro-phenoxy)-5-methyl-hexanoic acid;
(3S,5S)-3-Aminomethyl-6-(3-fluoro-phenoxy)-5-methyl-hexanoic acid;
(3S,5S)-3-Aminomethyl-6-(2-fluoro-phenoxy)-5-methyl-hexanoic acid;
(3S,5S)-3-Aminomethyl-6-(4-methoxy-phenoxy)-5-methyl-hexanoic acid;
(3S,5S)-3-Aminomethyl-6-(3-methoxy-phenoxy)-5-methyl-hexanoic acid;
(3S,5S)-3-Aminomethyl-6-(2-methoxy-phenoxy)-5-methyl-hexanoic acid;
(3S,5S)-3-Aminomethyl-5-methyl-6-(4-trifluoromethyl-phenoxy)-hexanoic acid;
(3S,5S)-3-Aminomethyl-5-methyl-6-(3-trifluoromethyl-phenoxy)-hexanoic acid;
(3S,5S)-3-Aminomethyl-5-methyl-6-(2-trifluoromethyl-phenoxy)-hexanoic acid;
(3S,5S)-3-Aminomethyl-5-methyl-6-(4-nitro-phenoxy)-hexanoic acid;
(3S,5S)-3-Aminomethyl-5-methyl-6-(3-nitro-phenoxy)-hexanoic acid;
(3S,5S)-3-Aminomethyl-5-methyl-6-(2-nitro-phenoxy)-hexanoic acid;
(3S,5S)-3-Aminomethyl-6-benzyloxy-5-methyl-hexanoic acid;
(3S,5S)-3-Aminomethyl-7-hydroxy-5-methyl-heptanoic acid;
(3S,5S)-3-Aminomethyl-7-methoxy-5-methyl-heptanoic acid;
(3S,5S)-3-Aminomethyl-7-ethoxy-5-methyl-heptanoic acid;
(3S,5S)-3-Aminomethyl-5-methyl-7-propoxy-heptanoic acid;
(3S,5S)-3-Aminomethyl-7-isopropoxy-5-methyl-heptanoic acid;
(3S,5S)-3-Aminomethyl-7-tert-butoxy-5-methyl-heptanoic acid;
(3S,5S)-3-Aminomethyl-7-fluoromethoxy-5-methyl-heptanoic acid;
(3S,5S)-3-Aminomethyl-7-(2-fluoro-ethoxy)-5-methyl-heptanoic acid;
(3S,5S)-3-Aminomethyl-5-methyl-7-(3,3,3-trifluoro-propoxy)-heptanoic acid;
(3S,5S)-3-Aminomethyl-7-benzyloxy-5-methyl-heptanoic acid;
(3S,5S)-3-Aminomethyl-5-methyl-7-phenoxy-heptanoic acid;
(3S,5S)-3-Aminomethyl-7-(4-chloro-phenoxy)-5-methyl-heptanoic acid;
(3S,5S)-3-Aminomethyl-7-(3-chloro-phenoxy)-5-methyl-heptanoic acid;
(3S,5S)-3-Aminomethyl-7-(2-chloro-phenoxy)-5-methyl-heptanoic acid;
(3S,5S)-3-Aminomethyl-7-(4-fluoro-phenoxy)-5-methyl-heptanoic acid;
(3S,5S)-3-Aminomethyl-7-(3-fluoro-phenoxy)-5-methyl-heptanoic acid;
(3S,5S)-3-Aminomethyl-7-(2-fluoro-phenoxy)-5-methyl-heptanoic acid;
(3S,5S)-3-Aminomethyl-7-(4-methoxy-phenoxy)-5-methyl-heptanoic acid;
(3S,5S)-3-Aminomethyl-7-(3-methoxy-phenoxy)-5-methyl-heptanoic acid;
(3S,5S)-3-Aminomethyl-7-(2-methoxy-phenoxy)-5-methyl-heptanoic acid;
(3S,5S)-3-Aminomethyl-5-methyl-7-(4-trifluoromethyl-phenoxy)-heptanoic acid;
(3S,5S)-3-Aminomethyl-5-methyl-7-(3-trifluoromethyl-phenoxy)-heptanoic acid;
(3S,5S)-3-Aminomethyl-5-methyl-7-(2-trifluoromethyl-phenoxy)-heptanoic acid;
(3S,5S)-3-Aminomethyl-5-methyl-7-(4-nitro-phenoxy)-heptanoic acid;
(3S,5S)-3-Aminomethyl-5-methyl-7-(3-nitro-phenoxy)-heptanoic acid;
(3S,5S)-3-Aminomethyl-5-methyl-7-(2-nitro-phenoxy)-heptanoic acid;
(3S,5S)-3-Aminomethyl-5-methyl-6-phenyl-hexanoic acid;
(3S,5S)-3-Aminomethyl-6-(4-chloro-phenyl)-5-methyl-hexanoic acid;
(3S,5S)-3-Aminomethyl-6-(3-chloro-phenyl)-5-methyl-hexanoic acid;
(3S,5S)-3-Aminomethyl-6-(2-chloro-phenyl)-5-methyl-hexanoic acid;
(3S,5S)-3-Aminomethyl-6-(4-methoxy-phenyl)-5-methyl-hexanoic acid;
(3S,5S)-3-Aminomethyl-6-(3-methoxy-phenyl)-5-methyl-hexanoic acid;
(3S,5S)-3-Aminomethyl-6-(2-methoxy-phenyl)-5-methyl-hexanoic acid;
(3S,5S)-3-Aminomethyl-6-(4-fluoro-phenyl)-5-methyl-hexanoic acid;
(3S,5S)-3-Aminomethyl-6-(3-fluoro-phenyl)-5-methyl-hexanoic acid;
(3S,5S)-3-Aminomethyl-6-(2-fluoro-phenyl)-5-methyl-hexanoic acid;
(3S,5R)-3-Aminomethyl-5-methyl-7-phenyl-heptanoic acid;
(3S,5R)-3-Aminomethyl-7-(4-chloro-phenyl)-5-methyl-heptanoic acid;
(3S,5R)-3-Aminomethyl-7-(3-chloro-phenyl)-5-methyl-heptanoic acid;
(3S,5R)-3-Aminomethyl-7-(2-chloro-phenyl)-5-methyl-heptanoic acid;
(3S,5R)-3-Aminomethyl-7-(4-methoxy-phenyl)-5-methyl-heptanoic acid;
(3S,5R)-3-Aminomethyl-7-(3-methoxy-phenyl)-5-methyl-heptanoic acid;
(3S,5R)-3-Aminomethyl-7-(2-methoxy-phenyl)-5-methyl-heptanoic acid;
(3S,5R)-3-Aminomethyl-7-(4-fluoro-phenyl)-5-methyl-heptanoic acid;
(3S,5R)-3-Aminomethyl-7-(3-fluoro-phenyl)-5-methyl-heptanoic acid;
(3S,5R)-3-Aminomethyl-7-(2-fluoro-phenyl)-5-methyl-heptanoic acid;
(3S,5S)-3-Aminomethyl-5-methyl-hept-6-enoic acid;
(3S,5R)-3-Aminomethyl-5-methyl-oct-7-enoic acid;
(3S,5R)-3-Aminomethyl-5-methyl-non-8-enoic acid;
(E)-(3S,5S)-3-Aminomethyl-5-methyl-oct-6-enoic acid;
(Z)-(3S,5S)-3-Aminomethyl-5-methyl-oct-6-enoic acid;
(Z)-(3S,5S)-3-Aminomethyl-5-methyl-non-6-enoic acid;
(E)-(3S,5S)-3-Aminomethyl-5-methyl-non-6-enoic acid;
(E)-(3S,5R)-3-Aminomethyl-5-methyl-non-7-enoic acid;
(Z)-(3 S,5R)-3-Aminomethyl-5-methyl-non-7-enoic acid;
(Z)-(3S,5R)-3-Aminomethyl-5-methyl-dec-7-enoic acid;
(E)-(3S,5R)-3-Aminomethyl-5-methyl-undec-7-enoic acid;
(3S,5S)-3-Aminomethyl-5,6,6-trimethyl-heptanoic acid;
(3S,5S)-3-Aminomethyl-5,6-dimethyl-heptanoic acid;
(3S,5S)-3-Aminomethyl-5-cyclopropyl-hexanoic acid;
(3S,5S)-3-Aminomethyl-5-cyclobutyl-hexanoic acid;
(3S,5S)-3-Aminomethyl-5-cyclopentyl-hexanoic acid;
(3S,5S)-3-Aminomethyl-5-cyclohexyl-hexanoic acid;
(3S,5R)-3-Aminomethyl-5-methyl-8-phenyl-octanoic acid;
(3S,5S)-3-Aminomethyl-5-methyl-6-phenyl-hexanoic acid;
(3S,5R)-3-Aminomethyl-5-methyl-7-phenyl-heptanoic acid;
(3R,4R,5R)-3-Aminomethyl-4,5-dimethyl-heptanoic acid; and
(3R,4R,5R)-3-Aminomethyl-4,5-dimethyl-octanoic acid.

38. The method according to claim 1, wherein the alpha2delta ligand is a compound of Formula (IXA) or Formula (IXB) 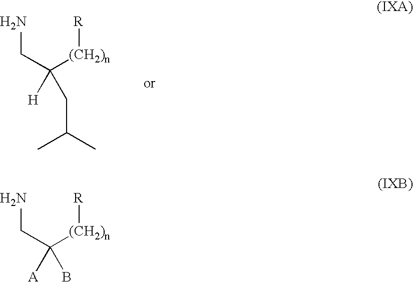
or a pharmaceutically acceptable salt thereof wherein:
n is an integer of from 0 to 2;
R is sulfonamide,
amide,
phosphonic acid,
heterocycle,
sulfonic acid, or
hydroxamic acid;
A is hydrogen or methyl; and 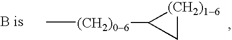
straight or branched alkyl of from 1 to 11 carbons, or —(CH₂)₁₋₄—Y—(CH₂)₀₋₄-phenyl wherein Y is —O—, —S—, —NR′₃ wherein
R′₃ is alkyl of from 1 to 6 carbons, cycloalkyl of from 3 to 8 carbons, benzyl or phenyl wherein benzyl or phenyl can be unsubstituted or substituted with from 1 to 3 substituents each independently selected from alkyl, alkoxy, halogen, hydroxy, carboxy, carboalkoxy, trifluoromethyl, and nitro.

39. The method according to claim 38, wherein R is a sulfonamide selected from —NHSO₂R¹⁵ and —SO₂NHR¹⁵, wherein R¹⁵ is straight or branched alkyl or trifluoromethyl.

40. The method according to claim 38, wherein R is a phosphonic acid, —PO₃H₂.

41. The method according to claim 38, wherein R is

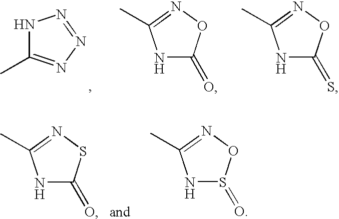

42. The method according to claim 38, wherein R is

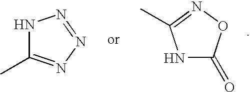

43. The method according to claim 38, wherein the compound of Formulas (IXA) or (IXB) is selected from:
4-Methyl-2-(1H-tetrazol-5-ylmethyl)-pentyl amine;
3-(2-Aminomethyl-4-methyl-pentyl)-4H-[1,2,4]oxadiazole-5-thione, HCl;
(2-Aminomethyl-4-methyl-pentyl)-phosphonic acid;
3-(3-Amino-2-cyclopentyl-propyl)-4H-[1,2,4]oxadiazol-5-one;
3-(3-Amino-2-cyclopentyl-propyl)-4H-[1,2,4]thiadiazol-5-one;
2-Cyclopentyl-3-(2-oxo-2,3-dihydro-2,4-[1,2,3,5]oxathiadiazol-4-yl)-propylamine;
3-(3-Amino-2-cyclobutyl-propyl)-4H-[1,2,4]oxadiazol-5-one;
3-(3-Amino-2-cyclobutyl-propyl)-4H-[1,2,4]thiadiazol-5-one; and
2-Cyclobutyl-3-(2-oxo-2,3-dihydro-2λ⁴-[1,2,3,5]oxathiadiazol-4-yl)-propylamine.

44. The method according to claim 38, wherein the alpha2delta ligand is 3-(2-aminomethyl-4-methyl-pentyl)-4H-[1,2,4]oxadiazol-5-one.

45. The method according to claim 38, wherein the alpha2delta ligand is 3-(2-aminomethyl-4-methyl-pentyl)-4H-[1,2,4]-oxadiazol-5-one hydrochloride.

46. The method according to claim 1, wherein the alpha2delta ligand is a compound of the Formula V, VI, VII, or VIII 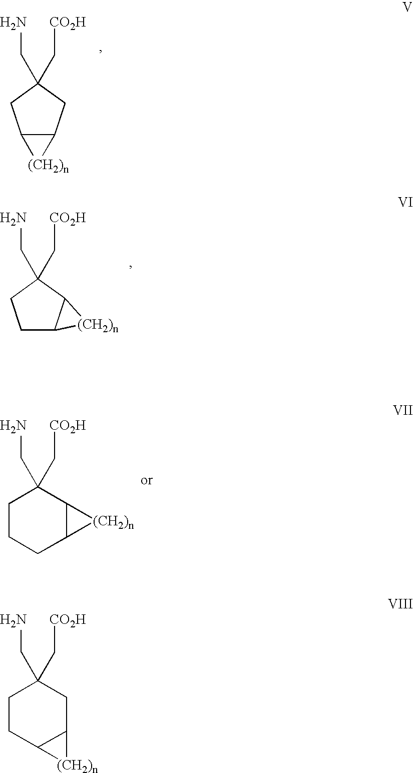
or pharmaceutically acceptable salt thereof,
wherein n is an integer of from 1 to 4, and
where there are stereocenters, each center may be independently R or S.

47. The method according to claim 46, wherein n is an integer of from 2 to 4.

48. The method according to claim 46, wherein the alpha2delta ligand is a compound of the Formula V.

49. The method according to claim 46, wherein the alpha2delta ligand is selected from:
(1α,6α,8β)(2-Aminomethy-octahydro-inden-2-yl)-acetic acid; (2-Aminomethyl-octahydro-inden-2-yl)-acetic acid; (2-Aminomethyl-octahydro-pentalen-2-yl)-acetic acid; (2-Aminomethyl-octahydro-pentalen-2-yl)-acetic acid; (3-Aminomethyl-bicyclo[3.2.0]hept-3-yl)-acetic acid; (3-Aminomethyl-bicyclo[3.2.0]hept-3-yl)-acetic acid; and (2-Aminomethyl-octahydro-inden-2-yl)-acetic acid.

50. The method according to claim 46, wherein the alpha2delta ligand is selected from:
(1α,5β)(3-Aminomethyl-bicyclo[3.1.0]hex-3-yl)-acetic acid,
(1α,5β)(3-Aminomethyl-bicyclo[3.2.0]hept-3-yl)-acetic acid,
(1α,5β)(2-Aminomethyl-octahydro-pentalen-2-yl)-acetic acid,
(1α,6β)(2-Aminomethyl-octahydro-inden-2-yl)-acetic acid,
(1α,7β)(2-Aminomethyl-decahydro-azulen-2-yl)-acetic acid,
(1α,5β)(3-Aminomethyl-bicyclo[3.1.0]hex-3-yl)-acetic acid,
(1α,5β)3-Aminomethyl-bicyclo[3.2.0]hept-3-yl)-acetic acid,
(1α,5β)(2-Aminomethyl-octahydro-pentalen-2-yl)-acetic acid,
(1α,6β)(2-Aminomethyl-octahydro-inden-2-yl)-acetic acid,
(1α,7β)(2-Aminomethyl-decahydro-azulen-2-yl)-acetic acid,
(1α,3α,5α)(3-Aminomethyl-bicyclo[3.1.0]hex-3-yl)-acetic acid,
(1α,3α,5α)(2-Aminomethyl-octahydro-pentalen-2-yl)-acetic acid,
(1α,6α,8α)(2-Aminomethyl-octahydro-inden-2-yl)-acetic acid,
(1α,7α,9α)(2-Aminomethyl-decahydro-azulen-2-yl)-acetic acid,
(1α,3β,5α)(3-Aminomethyl-bicyclo[3.1.0]hex-3-yl)-acetic acid,
(1α,3β,5α)(3-Aminomethyl-bicyclo[3.2.0]hept-3-yl)-acetic acid,
(1α,3β,5α)(2-Aminomethyl-octahydro-pentalen-2-yl)-acetic acid,
(1α,6α,8β)(2-Aminomethyl-octahydro-inden-2-yl)-acetic acid,
(1α,7α,9β)(2-Aminomethyl-decahydro-azulen-2-yl)-acetic acid,
((1R,3R,6R)-3-Aminomethyl-bicyclo[4.1.0]hept-3-yl)-acetic acid,
((1R,3S,6R)-3-Aminomethyl-bicyclo[4.1.0]hept-3-yl)-acetic acid,
((1S,3S,6S)-3-Aminomethyl-bicyclo[4.1.0]hept-3-yl)-acetic acid,
((1S,3R,6S)-3-Aminomethyl-bicyclo[4.1.0]oct-3-yl)-acetic acid,
((1R,3R,6S)-3-Aminomethyl-bicyclo[4.2.0]oct-3-yl)-acetic acid,
((1R,3S,6S)-3-Aminomethyl-bicyclo[4.2.0]oct-3-yl)-acetic acid,
((1S,3S,6R)-3-Aminomethyl-bicyclo[4.2.0]oct-3-yl)-acetic acid,
((1S,3R,6R)-3-Aminomethyl-bicyclo[4.2.0]oct-3-yl)-acetic acid,
((3αR,5R,7αS)-5-Aminomethyl-octahydro-inden-5-yl)-acetic acid,
((3αR,5S,7αS)-5-Aminomethyl-octahydro-inden-5-yl)-acetic acid,
((3αS,5S,7αR)-5-Aminomethyl-octahydro-inden-5-yl)-acetic acid,
((3αS,5R,7αR)-5-Aminomethyl-octahydro-inden-5-yl)-acetic acid,
((2R,4αS,8αR)-2-Aminomethyl-decahydro-naphthalen-2-yl)-acetic acid,
((2S,4αS,8αR)-2-Aminomethyl-decahydro-naphthalen-2-yl)-acetic acid,
((2S,4αR,8αS)-2-Aminomethyl-decahydro-naphthalen-2-yl)-acetic acid,
((2R,4αR,8αS)-2-Aminomethyl-decahydro-naphthalen-2-yl)-acetic acid,
((2R,4αS,9αR)-2-Aminomethyl-decahydro-benzocyclophepten-2-yl)acetic acid,
((2S,4αS,9αR)-2-Aminomethyl-decahydro-benzocyclophepten-2-yl) acetic acid,
((2S,4αR,9αS)-2-Aminomethyl-decahydro-benzocyclophepten-2-yl) acetic acid,
((2R,4αR,9αS)-2-Aminomethyl-decahydro-benzocyclophepten-2-yl) acetic acid,
((1R,3R,6S)-3-Aminomethyl-bicyclo[4.1.0]hept-3-yl)-acetic acid,
((1R,3S,6S)-3-Aminomethyl-bicyclo[4.1.0]hept-3-yl)-acetic acid,
((1S,3S,6R)-3-Aminomethyl-bicyclo[4.1.0]hept-3-yl)-acetic acid,
((1S,3R,6R)-3-Aminomethyl-bicyclo[4.1.0]hept-3-yl)-acetic acid,
((1R,3R,6R)-3-Aminomethyl-bicyclo[4.2.0]oct-3-yl)-acetic acid,
((1R,3S,6R)-3-Aminomethyl-bicyclo[4.2.0]oct-3-yl)-acetic acid,
((1S,3S,6S)-3-Aminomethyl-bicyclo[4.2.0]oct-3-yl)-acetic acid,
((1S,3R,6S)-3-Aminomethyl-bicyclo[4.2.0]oct-3-yl)-acetic acid,
((3αR,5R,7αR)-5-Aminomethyl-octahydro-inden-5-yl)-acetic acid,
((3αR,5S,7αR)-5-Aminomethyl-octahydro-inden-5-yl)-acetic acid,
((3αS,5S,7αS)-5-Aminomethyl-octahydro-inden-5-yl)-acetic acid,
((3αS,5R,7αS)-5-Aminomethyl-octahydro-inden-5-yl)-acetic acid,
((2R,4αR,8αR)-2-Aminomethyl-decahydro-naphthalen-2-yl)-acetic acid,
((2S,4αS,8αR)-2-Aminomethyl-decahydro-naphthalen-2-yl)-acetic acid,
((2S,4αR,8αS)-2-Aminomethyl-decahydro-naphthalen-2-yl)-acetic acid,
((2R,4αS,8αS)-2-Aminomethyl-decahydro-naphthalen-2-yl)-acetic acid,
((2R,4αR,90αR)-2-Aminomethyl-decahydro-benzocyclophepten-2-yl)-acetic acid,
((2S,4αR,9αR)-2-Aminomethyl-decahydro-benzocyclophepten-2-yl)-acetic acid,
((2S,4αS,9αS)-2-Aminomethyl-decahydro-benzocyclophepten-2-yl)-acetic acid, and
((2R,4αS,9αS)-2-Aminomethyl-decahydro-benzocyclophepten-2-yl)-acetic acid.

51. The method according to claim 46, wherein the alpha2delta ligand is (1α,3α,5α)(3-amino-methyl-bicyclo[3.2.0]hept-3-yl)-acetic acid.

52. The method according to claim 46, wherein the alpha2delta ligand is (1α,3α,5α)(3-aminomethyl-bicyclo[3.2.0.]hept-3-yl)-acetic acid hydrochloride.

53. The method according to claim 1, wherein the alpha2delta ligand is a compound of the Formula (XII) or (XIII) 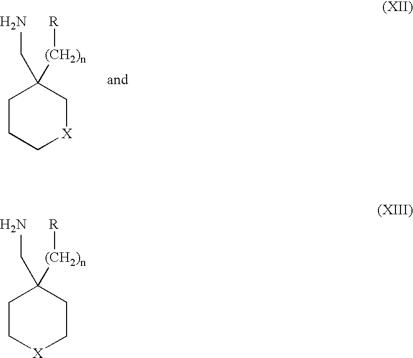
or a pharmaceutically acceptable salt thereof wherein:
n is an integer of from 0 to 2;
R is sulfonamide,
amide,
phosphonic acid,
heterocycle,
sulfonic acid, or
hydroxamic acid; and
X is —O—, —S—, —S(O)—, —S(O)₂—, or NR′₁ wherein R′₁ is hydrogen, straight or branched alkyl of from 1 to 6 carbons, benzyl, —C(O)R′₂ wherein R′₂ is straight or branched alkyl of 1 to 6 carbons, benzyl or phenyl or —CO₂R′₃ wherein R′₃ is straight or branched alkyl of from 1 to 6 carbons, or benzyl wherein the benzyl or phenyl groups can be unsubstituted or substituted by from 1 to 3 substituents selected from halogen, trifluoromethyl, and nitro.

54. The method according to claim 1, wherein the alpha2delta ligand is a compound of the Formula 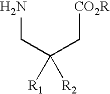
or a pharmaceutically acceptable salt thereof wherein:
R is hydrogen or lower alkyl;
R₁ is hydrogen or lower alkyl; 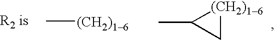
straight or branched alkyl of from 7 to 11 carbon atoms, or —(CH₂)₍₁₋₄₎—X—(CH₂)₍₀₋₄₎-phenyl wherein
X is —O—, —S—, —NR₃ wherein
R₃ is alkyl of from 1 to 6 carbons, cycloalkyl of from 3 to 8 carbons, benzyl or phenyl;
wherein phenyl and benzyl can be unsubstituted or substituted with from 1 to 3 substituents each independently selected from alkyl, alkoxy, halogen, hydroxy, carboxy, carboalkoxy, trifluoromethyl, amino, and nitro.

55. The method according to claim 1, wherein the alpha2delta ligand is a compound of the Formula (1), (2), (3), (4), (5), (6), (7), or (8) 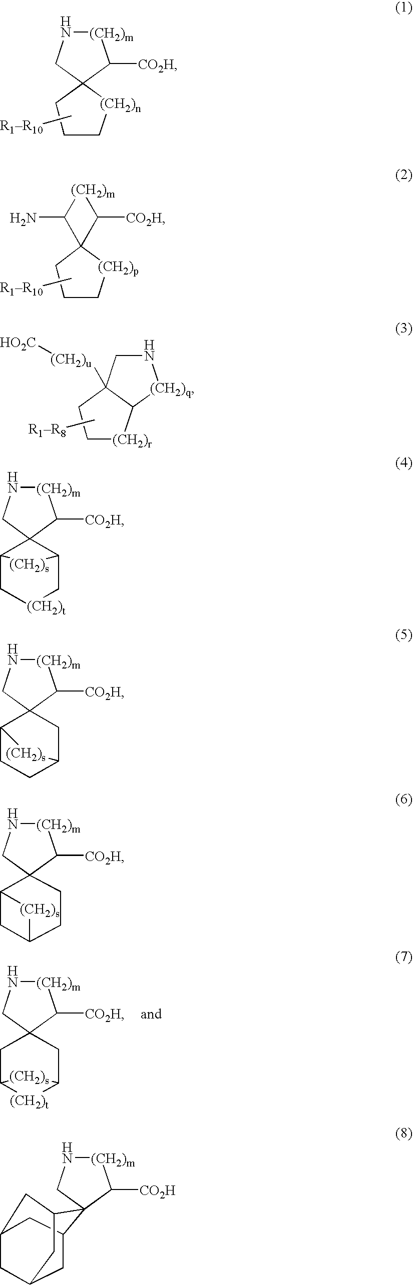
or a pharmaceutically acceptable salt thereof or a prodrug thereof wherein:
R₁ to R₁₀ are each independently selected from hydrogen or a straight or branched alkyl of from 1 to 6 carbons, benzyl, or phenyl;
m is an integer of from 0 to 3;
n is an integer of from 1 to 2;
o is an integer of from 0 to 3;
p is an integer of from 1 to 2;
q is an integer of from 0 to 2;
r is an integer of from 1 to 2;
s is an integer of from 1 to 3;
t is an integer of from 0 to 2; and
u is an integer of from 0 to 1.
